(19) United States
(12) Patent Application Publication
(10) Pub. No.: US 2019/0112671 A1
(43) Pub. Date: Apr. 18, 2019

(54) Breast Cancer Immunotherapy and Methods

(71) Applicant: Florida State University Research Foundation, Inc., Tallahassee, FL (US)

(72) Inventors: Jinfeng Zhang, Tallahassee, FL (US); Qing-Xiang Amy Sang, Tallahassee, FL (US); Mayassa Bou Dargham, Tallahassee, FL (US); Yuhang Liu, Tallahassee, FL (US)

(21) Appl. No.: 16/205,871

(22) Filed: Nov. 30, 2018

Related U.S. Application Data
(60) Provisional application No. 62/593,483, filed on Dec. 1, 2017.

(51) Int. Cl. C12Q 1/6886

(52) U.S. Cl. CPC C12Q 1/6886; C12Q 2600/112; C12Q 2600/106; C12Q 2600/158

(57) ABSTRACT

Methods of treating a patient, such as a patient with breast cancer, and methods of selecting a therapy for a patient. The methods of treating a patient may include determining one or more expression levels for a set of biomarkers from a biological sample of the patient. The biological sample may include breast cancer tissue.

### CROSS-REFERENCE TO RELATED APPLICATIONS

This application claims priority to U.S. Provisional Patent Application No. 62/593,483, filed Dec. 1, 2017, which is incorporated herein by reference.

### BACKGROUND

Cancer cells express markers that differentiate them from normal cells and may allow for their detection through immune surveillance and subsequent destruction. The recognition of antigen-MHC-I complexes by naïve cytotoxic T lymphocytes (CTLs; aka CD8⁺ T cells) can result in their activation. Then, trafficking and infiltration of CTLs from the bloodstream to the tumor microenvironment follows, orchestrated by adhesion molecules. Activated CTLs can kill cancer cells via (i) granular exocytosis: perforins and granzymes, or (ii) death ligand-death receptor-mediated apoptosis. The latter occurs through the binding of death ligand cluster of differentiation 95 (CD95L; aka FasL) to the death receptor CD95 (Fas) on the tumor cell surface. This process, from antigen presentation to apoptosis induction by CTL, is known as the cancer-immunity cycle.

Despite immune surveillance, cancer cells typically manage to evade immune destruction. There are multitudes of evasion mechanisms that may govern the suppressive nature of the tumor microenvironment and the analysis of these mechanisms is an active area of study. Given the heterogeneity of breast cancer, several studies have been conducted to investigate the molecular and prognostic differences between the different hormone receptor subtypes and the ductal and lobular subgroups. Triple-negative breast cancer (TNBC) studies have identified intra-subtype heterogeneity where certain patients showed poor prognosis while others responded well to anthracycline-based treatments (e.g., Stagg, J. et al., Ther. Adv. Med. Oncol. 2013; 5:169-81).

Scientific evidence suggested that the clinical outcome for TNBC hormone receptor subtype was affected by tumor-infiltrating immune cells (Id.). Studies on invasive breast cancer showed that invasive lobular carcinoma (ILC) is 3 times more likely to metastasize and less responsive to neoadjuvant chemotherapy compared to invasive ductal carcinoma (IDC) (Korkola, J. E., et al. Cancer Res. 2003; 63:7167-75). Furthermore, an “immune-related” subgroup of ILC was identified and characterized by upregulated mRNA expression of PD-1, it's ligand PD-L1, and CTLA4 (Michaut, M. et al., Sci. Rep. 2016; 6).

Currently, immune checkpoints, CTLA4 and PD-1/PD-L1, are the most intensively studied immune evasion molecules in cancer, along with the immunotherapies targeting them. However, substantial proportions of PD-L1 positive or CTLA4 positive patients do not respond to the corresponding immunotherapies (e.g., Vonderheide, R. H., et al., Clin. Cancer Res. 2010; 16:3485-94; and Mittendorf, E. A. et al., Am. J. Hematol. 2015; 11). Thus, these molecules generally are not reliable biomarkers for the prediction of treatment response (Socinski, M. et al., Ann. Oncol. 2016; 27).

Furthermore, clinical trials generally appear to be shifting towards combination immunotherapy, likely under the assumption that multiple immune evasion mechanisms may be utilized by a tumor. However, the choice of immunotherapy and combined treatments is generally poorly guided as they are typically given indiscriminately even though different patients may have different evasion mechanisms.

In view of the differential immune responses and/or evasion mechanisms in breast cancer, there remains a need for identifying different breast cancers based on their expression of immune-related genes. There also remains a need for improved treatment methods.

### BRIEF SUMMARY

Described herein are methods of sequential biclustering of The Cancer Genome Atlas RNAseq breast cancer data, and the identification of 7 clusters. Also provided are methods of treatment based on patients' response to immunotherapies. The methods of treatment may include administering combination therapies that may be rationally designed in view of the testing described herein.

In one aspect, methods of treating a patient are provided. In some embodiments, the methods include determining one or more expression levels for a set of biomarkers from a biological sample of the patient; determining an immune evasion subtype for the biological sample based on the one or more expression levels; selecting a treatment based on the immune evasion subtype; and administering the treatment to the patient.

In some embodiments, the set of biomarkers includes interleukin-2 receptor subunit gamma (IL2RG). The set of biomarkers also may include ATP-binding cassette sub-family B member 1 (ABCB1) and/or decorin (DCN). In some embodiments, the set of biomarkers also includes lymphocyte-specific protein tyrosine kinase (LCK) and/or estrogen receptor-1 (ESR1). In some embodiments, the set of biomarkers also includes one or more of selectin-P (SELP), glucose-6-phosphate dehydrogenase (G6PD), programmed cell death-1 (PD-1), cluster of differentiation-3 subunit gamma (CD3G), B-Cell CLL/Lymphoma 2 (BCL2)-associated X Protein (BAX), C—C chemokine receptor type 5 (CCR5), or cluster of differentiation-40 ligand (CD40LG).

In some embodiments, the set of biomarkers includes estrogen receptor-1 (ESR1). In some embodiments, the set of biomarkers includes estrogen receptor-1 (ESR1) and interleukin-2 receptor subunit gamma (IL2RG). In some embodiments, the set of biomarkers includes at least one of estrogen receptor-1 (ESR1) and interleukin-2 receptor subunit gamma (IL2RG); and one or more of selectin-P (SELP), signaling lymphocytic activation molecule family member 1 (SLAMF1), lymphocyte-specific protein tyrosine kinase (LCK), cluster of differentiation 2 (CD2), or casein kappa protein coding gene (CSN3). In some embodiments, the set of biomarkers includes at least one of estrogen receptor-1 (ESR1) and interleukin-2 receptor subunit gamma (IL2RG); one or more of selectin-P (SELP), signaling lymphocytic activation molecule family member 1 (SLAMF1), lymphocyte-specific protein tyrosine kinase (LCK), cluster of differentiation 2 (CD2), or casein kappa protein coding gene (CSN3); and one or more of ATP-binding cassette sub-family B member 1 (ABCB1), decorin (DCN), cluster of differentiation-3 subunit gamma (CD3G), cluster of differentiation 5 (CD5), granzyme A (GZMA), or granzyme B (GZMB). In some embodiments, the set of biomarkers includes at least one of estrogen receptor-1 (ESR1) and interleukin-2 receptor subunit gamma (IL2RG); one or more of selectin-P (SELP), signaling lymphocytic activation molecule family member 1 (SLAMF1), lymphocyte-specific protein tyrosine kinase (LCK), cluster of differentiation 2 (CD2), or casein kappa protein coding gene (CSN3); one or more of ATP-binding cassette sub-family B member 1 (ABCB1), decorin (DCN), cluster of differentiation-3 subunit gamma (CD3G), cluster of differentiation 5 (CD5), granzyme A (GZMA), or granzyme B (GZMB); and one or more of cluster of differentiation-40 ligand (CD40LG), glucose-6-phosphate dehydrogenase (G6PD), programmed cell death-1 (PD-1), T-cell receptor T3 delta chain (CD3D), B-Cell CLL/Lymphoma 2 (BCL2)-associated X Protein (BAX), or C—C chemokine receptor type 5 (CCR5). In some embodiments, the set of biomarkers includes at least one of estrogen receptor-1 (ESR1) and interleukin-2 receptor subunit gamma (IL2RG); one or more of selectin-P (SELP), signaling lymphocytic activation molecule family member 1 (SLAMF1), lymphocyte-specific protein tyrosine kinase (LCK), cluster of differentiation 2 (CD2), or casein kappa protein coding gene (CSN3); one or more of ATP-binding cassette sub-family B member 1 (ABCB1), decorin (DCN), cluster of differentiation-3 subunit gamma (CD3G), cluster of differentiation 5 (CD5), granzyme A (GZMA), or granzyme B (GZMB); one or more of cluster of differentiation-40 ligand (CD40LG), glucose-6-phosphate dehydrogenase (G6PD), programmed cell death-1 (PD-1), T-cell receptor T3 delta chain (CD3D), B-Cell CLL/Lymphoma 2 (BCL2)-associated X Protein (BAX), or C—C chemokine receptor type 5 (CCR5); and one or more of tumor protein p63 (TP63), bone marrow tyrosine kinase gene in chromosome X protein (BMX), polo like kinase 1 (PLK1), transforming growth factor beta 2 (TGFB2), NADH ubiquinone oxidoreductase subunit B9 (NDUFB9), transforming growth factor beta receptor associated protein 1 (TGFBRAP1), 5-hydroxytryptamine receptor 2A (HTR2A), PLAG1 like zinc finger 1 (PLAGL1), sprouty RTK signaling antagonist 2 (SPRY2), protein tyrosine phosphatase receptor type C (PTPRC), Fc receptor like 3 (FCRL3), protein kinase C beta (PRKCB), heat shock protein beta-6 (HSPB6), contactin 1 (CNTN1), crystallin alpha B (CRYAB), heat shock protein 90 alpha family class A member 1 (HSP90AA1), MAGE family member A1 (MAGEA1), or interferon regulatory factor 8 (IRF8).

In some embodiments, the set of biomarkers includes interleukin-2 receptor subunit gamma (IL2RG). In some embodiments, the set of biomarkers includes interleukin-2 receptor subunit gamma (IL2RG), and one or more of ATP-binding cassette sub-family B member 1 (ABCB1), signaling lymphocytic activation molecule family member 1 (SLAMF1), cluster of differentiation 2 (CD2), cluster of differentiation 5 (CD5), decorin (DCN), cluster of differentiation-3 subunit gamma (CD3G), selectin-P (SELP), or lymphocyte-specific protein tyrosine kinase (LCK). In some embodiments, the set of biomarkers includes interleukin-2 receptor subunit gamma (IL2RG); one or more of ATP-binding cassette sub-family B member 1 (ABCB1), signaling lymphocytic activation molecule family member 1 (SLAMF1), cluster of differentiation 2 (CD2), cluster of differentiation 5 (CD5), decorin (DCN), cluster of differentiation-3 subunit gamma (CD3G), selectin-P (SELP), or lymphocyte-specific protein tyrosine kinase (LCK); and one or more of T-cell receptor T3 delta chain (CD3D), signaling threshold-regulating transmembrane adapter 1 (SIT1), IL2 inducible T cell kinase (ITK), CD3e molecule (CD3E), Src like adaptor 2 (SLA2), CD247 molecule (CD247), or granzyme A (GZMA).

In another aspect, methods of selecting a therapy for a patient are provided. In some embodiments, the methods include determining expression levels for a set of biomarkers from a biological sample isolated from the patient; determining an immune evasion subtype for the biological sample based on the expression levels; and selecting the therapy based on the immune evasion subtype.

Additional aspects will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 depicts possible mechanisms of immunity evasion by cancer at different levels of the cancer-immunity cycle.

FIG. 2A, FIG. 2B, and FIG. 2C depict clustering results for an embodiment of sequential biclustering.

FIG. 3A, FIG. 3B, FIG. 3C, and FIG. 3D depict embodiments of a pathway analysis based on the log 2 fold change for clusters 1 and 4 compared to normal: fold change level of molecules involved in antigen processing and presentation molecules in cluster 1 (FIG. 3A) and cluster 4 (FIG. 3B); fold change level of molecules involved in leukocyte recruitment in cluster 1 (FIG. 3C) and cluster 4 (FIG. 3D).

FIG. 4 depicts an embodiment of a classification tree with 12 biomarkers and their log 2 gene expression cutoffs for the identified clusters (CL).

FIG. 5 depicts the frequency at which genes were used in an embodiment of a random forest test.

FIG. 6 depicts plots of mean decrease in accuracy and mean decrease in Gini index for the 20 genes of an embodiment of a random forest test.

### DETAILED DESCRIPTION

In some embodiments, a sequential biclustering method is used on The Cancer Genome Atlas RNAseq breast cancer data, which results in the identification of clusters (for example, 7 clusters), based, at least in part, on the expression of immune-related genes.

In some embodiments, about 77.4% of the clustered tumor specimens evade through tumor growth factor-beta (TGF-β) immunosuppression, 57.8% through decoy receptor 3 (DcR3) counterattack, 48.0% through cytotoxic T-lymphocyte-associated protein 4 (CTLA4), and 34.3% through programmed cell death-1 (PD-1). In some embodiments, TGF-β and/or DcR3 are drug targets for breast cancer immunotherapy. Targeting TGF-β and/or DcR3 may provide a powerful approach for treating breast cancer, at least because 57.8% of patients, in some embodiments, overexpressed these two drug targets.

Furthermore, in some embodiments, triple-negative breast cancer (TNBC) patients cluster equally into two subgroups: one with impaired antigen presentation and another with high leukocyte recruitment and four different evasion mechanisms. Therefore, in some embodiments, TNBC patients are treated with different immunotherapy approaches. Also identified are biomarkers to cluster patients into subgroups based on immune evasion mechanisms in order to guide the choice of immunotherapy.

The methods provided herein generally may include treating a patient. The patient may have one or more types of cancer, including, but not limited to, breast cancer. For example, the patient may have breast cancer, prostate cancer, colon cancer, lung cancer, a head/neck cancer, a bone cancer, a blood cancer, or a combination thereof. A patient is “treated” when a treatment is administered to the patient, wherein the “treatment” eliminates and/or reverses, stops, and/or slows the growth and/or spread of a disease, such as cancer.

In some embodiments, the methods include determining one or more expression levels for a set of biomarkers from a biological sample of the patient; determining an immune evasion subtype for the biological sample based on the one or more expression levels; selecting a treatment based on the immune evasion subtype; and administering the treatment to the patient.

In some embodiments, the set of biomarkers includes interleukin-2 receptor subunit gamma (IL2RG), ATP-binding cassette sub-family B member 1 (ABCB1), and decorin (DCN). In some embodiments, the set of biomarkers includes interleukin-2 receptor subunit gamma (IL2RG), ATP-binding cassette sub-family B member 1 (ABCB1), decorin (DCN), lymphocyte-specific protein tyrosine kinase (LCK), and estrogen receptor-1 (ESR1).

In some embodiments, the set of biomarkers includes interleukin-2 receptor subunit gamma (IL2RG), ATP-binding cassette sub-family B member 1 (ABCB1), decorin (DCN), lymphocyte-specific protein tyrosine kinase (LCK), estrogen receptor-1 (ESR1), selectin-P (SELP), glucose-6-phosphate dehydrogenase (G6PD), programmed cell death-1 (PD-1), cluster of differentiation-3 subunit gamma (CD3G), B-Cell CLL/Lymphoma 2 (BCL2)-associated X Protein (BAX), C—C chemokine receptor type 5 (CCR5), and cluster of differentiation-40 ligand (CD40LG).

In some embodiments, the set of biomarkers includes interleukin-2 receptor subunit gamma (IL2RG). In some embodiments, the set of biomarkers includes interleukin-2 receptor subunit gamma (IL2RG), and one or more of ATP-binding cassette sub-family B member 1 (ABCB1) or decorin (DCN). In some embodiments, the set of biomarkers also includes lymphocyte-specific protein tyrosine kinase (LCK) and estrogen receptor-1 (ESR1). In some embodiments, the set of biomarkers also includes selectin-P (SELP), glucose-6-phosphate dehydrogenase (G6PD), programmed cell death-1 (PD-1), cluster of differentiation-3 subunit gamma (CD3G), B-Cell CLL/Lymphoma 2 (BCL2)-associated X Protein (BAX), C—C chemokine receptor type 5 (CCR5), or cluster of differentiation-40 ligand (CD40LG).

In some embodiments, the set of biomarkers includes estrogen receptor-1 (ESR1).

In some embodiments, the set of biomarkers includes estrogen receptor-1 (ESR1) and interleukin-2 receptor subunit gamma (IL2RG).

In some embodiments, the set of biomarkers includes at least one of estrogen receptor-1 (ESR1) and interleukin-2 receptor subunit gamma (IL2RG); and one or more of selectin-P (SELP), signaling lymphocytic activation molecule family member 1 (SLAMF1), lymphocyte-specific protein tyrosine kinase (LCK), cluster of differentiation 2 (CD2), or casein kappa protein coding gene (CSN3).

In some embodiments, the set of biomarkers includes at least one of estrogen receptor-1 (ESR1) and interleukin-2 receptor subunit gamma (IL2RG); one or more of selectin-P (SELP), signaling lymphocytic activation molecule family member 1 (SLAMF1), lymphocyte-specific protein tyrosine kinase (LCK), cluster of differentiation 2 (CD2), or casein kappa protein coding gene (CSN3); and one or more of ATP-binding cassette sub-family B member 1 (ABCB1), decorin (DCN), cluster of differentiation-3 subunit gamma (CD3G), cluster of differentiation 5 (CD5), granzyme A (GZMA), or granzyme B (GZMB).

In some embodiments, the set of biomarkers includes at least one of estrogen receptor-1 (ESR1) and interleukin-2 receptor subunit gamma (IL2RG); one or more of selectin-P (SELP), signaling lymphocytic activation molecule family member 1 (SLAMF1), lymphocyte-specific protein tyrosine kinase (LCK), cluster of differentiation 2 (CD2), or casein kappa protein coding gene (CSN3); one or more of ATP-binding cassette sub-family B member 1 (ABCB1), decorin (DCN), cluster of differentiation-3 subunit gamma (CD3G), cluster of differentiation 5 (CD5), granzyme A (GZMA), or granzyme B (GZMB); and one or more of cluster of differentiation-40 ligand (CD40LG), glucose-6-phosphate dehydrogenase (G6PD), programmed cell death-1 (PD-1), T-cell receptor T3 delta chain (CD3D), B-Cell CLL/Lymphoma 2 (BCL2)-associated X Protein (BAX), or C—C chemokine receptor type 5 (CCR5).

In some embodiments, the set of biomarkers includes at least one of estrogen receptor-1 (ESR1) and interleukin-2 receptor subunit gamma (IL2RG); one or more of selectin-P (SELP), signaling lymphocytic activation molecule family member 1 (SLAMF1), lymphocyte-specific protein tyrosine kinase (LCK), cluster of differentiation 2 (CD2), or casein kappa protein coding gene (CSN3); one or more of ATP-binding cassette sub-family B member 1 (ABCB1), decorin (DCN), cluster of differentiation-3 subunit gamma (CD3G), cluster of differentiation 5 (CD5), granzyme A (GZMA), or granzyme B (GZMB); one or more of cluster of differentiation-40 ligand (CD40LG), glucose-6-phosphate dehydrogenase (G6PD), programmed cell death-1 (PD-1), T-cell receptor T3 delta chain (CD3D), B-Cell CLL/Lymphoma 2 (BCL2)-associated X Protein (BAX), or C—C chemokine receptor type 5 (CCR5); and one or more of tumor protein p63 (TP63), bone marrow tyrosine kinase gene in chromosome X protein (BMX), polo like kinase 1 (PLK1), transforming growth factor beta 2 (TGFB2), NADH ubiquinone oxidoreductase subunit B9 (NDUFB9), transforming growth factor beta receptor associated protein 1 (TGFBRAP1), 5-hydroxytryptamine receptor 2A (HTR2A), PLAG1 like zinc finger 1 (PLAGL1), sprouty RTK signaling antagonist 2 (SPRY2), protein tyrosine phosphatase receptor type C (PTPRC), Fc receptor like 3 (FCRL3), protein kinase C beta (PRKCB), heat shock protein beta-6 (HSPB6), contactin 1 (CNTN1), crystallin alpha B (CRYAB), heat shock protein 90 alpha family class A member 1 (HSP90AA1), MAGE family member A1 (MAGEA1), or interferon regulatory factor 8 (IRF8).

In some embodiments, the set of biomarkers includes interleukin-2 receptor subunit gamma (IL2RG).

In some embodiments, the set of biomarkers includes interleukin-2 receptor subunit gamma (IL2RG), and one or more of ATP-binding cassette sub-family B member 1 (ABCB1), signaling lymphocytic activation molecule family member 1 (SLAMF1), cluster of differentiation 2 (CD2), cluster of differentiation 5 (CD5), decorin (DCN), cluster of differentiation-3 subunit gamma (CD3G), selectin-P (SELP), or lymphocyte-specific protein tyrosine kinase (LCK). In some embodiments, the set of biomarkers includes interleukin-2 receptor subunit gamma (IL2RG); one or more of ATP-binding cassette sub-family B member 1 (ABCB1), signaling lymphocytic activation molecule family member 1 (SLAMF1), cluster of differentiation 2 (CD2), cluster of differentiation 5 (CD5), decorin (DCN), cluster of differentiation-3 subunit gamma (CD3G), selectin-P (SELP), or lymphocyte-specific protein tyrosine kinase (LCK); and one or more of T-cell receptor T3 delta chain (CD3D), signaling threshold-regulating transmembrane adapter 1 (SIT1), IL2 inducible T cell kinase (ITK), CD3e molecule (CD3E), Src like adaptor 2 (SLA2), CD247 molecule (CD247), and granzyme A (GZMA).

In some embodiments, the set of biomarkers includes one or more of IL2RG, ABCB1, SLAMF1, CD2, DCN, CD3G, SELP, LCK, GZMA, or GZMB.

In some embodiments, the set of biomarkers includes one or more of IL2RG, ABCB1, CD40LG, DCN, SELP, LCK, ESR1, G6PD, PDCD1, BAX, CD3G, CCR5, SLAMF1, TP63, GZMB, CD2, CD5, or CSN3.

In some embodiments, the set of biomarkers includes one or more of IL2RG, CD3D, SIT1, SLAMF1, CD2, CD5, ITK, LCK, DCN, CD3E, SELP, SLA2, CD3G, CD247, ABCB1, or GZMA.

In some embodiments, the treatment that is administered to the patient includes one or more agents. Essentially any agent may be administered to the patient using any known devices and/or delivery methods. For example, the administering of the treatment may include injecting the treatment intravenously.

The one or more agents may include a drug. The term “drug” as used herein encompasses any suitable pharmaceutically active ingredient. The drug may be small molecule, macromolecule, biologic, or metabolite, among other forms/types of active ingredients. The drug described herein includes its alternative forms, such as salt forms, free acid forms, free base forms, and hydrates. The drug may be formulated with one or more pharmaceutically acceptable excipients known in the art.

In some embodiments, the treatment includes one or more agents selected from anti-PDCD1 agent, an anti-cytotoxic T-lymphocyte-associated protein 4 (anti-CTLA4) agent, ex vivo modulated dendritic cells, chimeric antigen receptor T cells (CAR-Ts), an anti-transforming growth factor beta 1 (TGFβ-1) agent, an anti-decoy receptor 3 (DcR3) agent, an anti-interferon gamma (IFN-γ) agent, or a combination thereof.

In some embodiments, the treatment includes an immunotherapy agent. The immunotherapy agent may be selected from anti-TGF-β1, anti-PD-1, anti-CTLA4, anti-DcR3, anti-IFN-γ*, ex-vivo modulated dendritic cells, or a combination thereof.

The biological sample may include any tissue, fluid, or a combination thereof collected from a patient. For example, the biological tissue may include blood, cancer tissue, or a combination thereof. A cancer tissue generally may include a tumor tissue. In some embodiments, the biological tissue includes breast cancer tissue.

In some embodiments, the methods herein include selecting a therapy for a patient with breast cancer. The methods may include determining expression levels for a set of biomarkers from a biological sample isolated from the patient, wherein the set of biomarkers may include any of those described herein; determining an immune evasion subtype for the biological sample based on the expression levels; and selecting the therapy based on the immune evasion subtype.

While certain aspects of conventional technologies have been discussed to facilitate disclosure of various embodiments, applicants in no way disclaim these technical aspects, and it is contemplated that the present disclosure may encompass one or more of the conventional technical aspects discussed herein.

In the descriptions provided herein, the terms “includes,” “is,” “containing,” “having,” and “comprises” are used in an open-ended fashion, and thus should be interpreted to mean “including, but not limited to.” When methods are claimed or described in terms of “comprising” various components or steps, the methods can also “consist essentially of” or “consist of” the various components or steps, unless stated otherwise.

The terms “a,” “an,” and “the” are intended to include plural alternatives, e.g., at least one. For instance, the disclosure of “a biomarker,” “an immune evasion mechanism,” “a biological sample”, and the like, are meant to encompass one, or mixtures or combinations of more than one biomarker, immune evasion mechanism, biological sample, and the like, unless otherwise specified.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### Example 1—Subgrouping Breast Cancer Patients Based on Immune Evasion Mechanisms

In this example, subgrouping breast cancer patients based on immune evasion mechanisms revealed a high involvement of tumor growth factor-beta and decoy receptor. Specifically, the tests of this example were configured to provide further insight into immune evasion mechanisms and heterogeneity in breast cancer and provide a better diagnostic to help guide treatments.

The sequential biclustering method was used, and a classification tree algorithm was used to analyze the expression pattern of The Cancer Genome Atlas (TCGA) RNA-seq data of immune-related genes and identify putative biomarkers, respectively. Seven distinct breast cancer groups were identified that represent different combinations of evasion mechanisms (M) and reveal molecular features that may provide a better understanding of the evasion mechanisms in breast cancer and suggest potential therapeutic strategies.

The materials and methods of this example are explained in the following paragraphs.

Generating the List of Immune Genes:

Based on the current knowledge of the mechanism of tumor evasion from immune system destruction, a list of 87 genes was generated manually based on the available literature.

| | |
|---|---|
| Number of patients (%) | Number of genes |

| | | | |
|---|---|---|---|
| Cluster 1 | 296 | (27.8%) | 145 |
| Cluster 2 | 87 | (8.2%) | 147 |
| Cluster 3 | 143 | (13.4%) | 127 |
| Cluster 4 | 108 | (10.1%) | 97 |
| Cluster 5 | 111 | (10.4%) | 78 |
| Cluster 6 | 60 | (5.6%) | 113 |
| Cluster 7 | 59 | (5.5%) | 71 |
| total | 864 | (81.1%) | 778 |

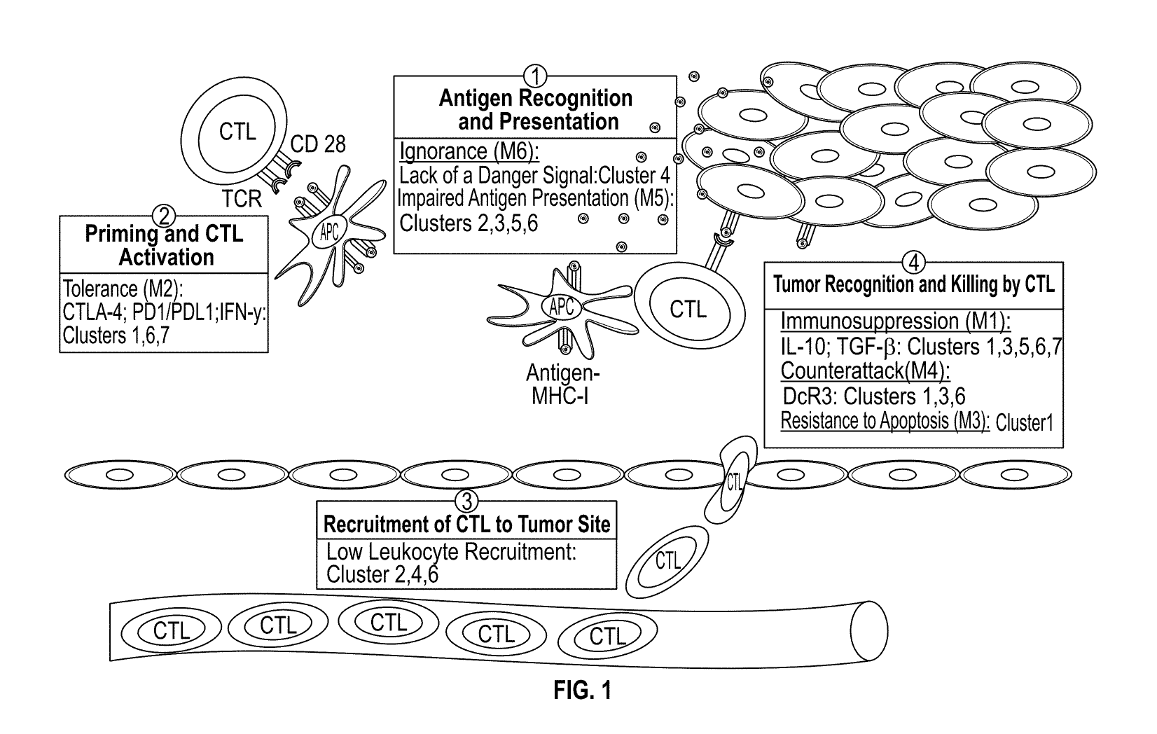

In order to take no less than about 5% of all patients, Clusters with no less than 50 patients were chosen. The clusters selected in each iteration made up the final set of clusters. The generated list included genes involved in the cancer-immunity cycle, and tolerance and immunosuppression-inducing genes (FIG. 1). FIG. 1 depicts evasion at different levels of the cancer-immunity cycle in each cluster.

To make sure no important genes were missing, the list was expanded from 87 genes to 1,356 by adding all interacting proteins determined using a bioGRID database (Stark, C. et al., “BioGRID: a general repository for interaction datasets,” Nucleic Acids Res 2006; 34:D535-9. doi:10.1093/nar/gkj109).

Full List of Immune-Related Genes from Protein-Protein Interaction (1,356 Genes):

ABCB1, ABL1, ABL2, ABP1, ACP1, ACTA1, ACTB, ACTG1, ACTL6A, ACTR1A, ACTR1B, ACVRL1, ADAP2, AES, AGAP2, AGTR1, AGT, AGXT, AIMP2, AKT1, ALDOA, ALK, AMFR, ANG, ANK2, ANKRD2, AP1B1, AP1M1, AP2A1, AP2B1, AP2M1, APCS, APEX1, APEX2, APOA1, APOH, APP, APTX, AREG, ARG1, ARHGAP15, ARHGAP9, ARHGDIA, ARHGEF1, ARHGEF7, ARID1A, ARID3A, ARIH2, ARL3, ARPP19, ARRB1, AR, ASB2, ATF3, ATG3, ATM, ATP5C1, ATP5F1, ATPSH, ATP6V0A1, ATP6V0D1, ATP6V1A, ATP6V1B2, ATR, AURKA, AURKB, AXIN1, B2M, BACH1, BAD, BAG1, BAG4, BAG5, BAIAP2L1, BAK1, BANP, BARD1, BAT3, BAX, BCAP31, BCAS1, BCL10, BCL2L1, BCL2, BDKRB2, BGN, BID, BIRC2, BIRC3, BLM, BMI1, BMP1, BMX, BRAF, BRAP, BRCA1, BRCA2, BRCC3, BRD1, BRE, BRF1, BRIP1, BRPF1, BRSK1, BST2, BTK, BTRC, C6orf134, C6orf89, C6, CABLES1, CABLES2, CACNB3, CACNB4, CALM1, CALR, CAMK2A, CAMK2B, CAMK2D, CANX, CAPZA2, CARM1, CASKIN1, CASP10, CASP2, CASP3, CASP7, CASP8AP2, CASP8, CAV1, CBLB, CBLC, CBL, CBX4, CCAR1, CCDC8, CCK, CCL22, CCNA2, CCNG1, CCNH, CONI, CCR4, CCR5, CD14, CD1D, CD22, CD247, CD24, CD274, CD28, CD2AP, CD2, CD38, CD3D, CD3EAP, CD3E, CD3G, CD40LG, CD40, CD44, CD47, CD4, CD5, CD79A, CD79B, CD7, CD80, CD86, CD8A, CDC14A, CDC14B, CDC42EP2, CDC42EP3, CDC42SE1, CDC42, CDK1, CDK2, CDK5, CDK7, CDK8, CDK9, CDKN1A, CDKN1B, CDKN2A, CDS1, CEBPZ, CEP110, CFLAR, CHCHD3, CHD3, CHD8, CHEK1, CHEK2, CHUK, CISH, CLIP3, CLSTN1, CLTC, CNOT6, CNP, CNTN1, COG6, COL2A1, COPB1, COPG, COPS2, COPS3, COPS5, COPS7A, COX5A, COX5B, COX6A1, COX6B1, COX6C, COX7A2L, COX7A2, CRADD, CREB1, CREBBP, CRK, CRYAB, CSF2RA, CSF2RB, CSF2, CSF3R, CSN2, CSN3, CSNK1A1, CSNK1D, CSNK2A1, CSNK2A2, CSTA, CTBP1, CTCF, CTDP1, CTLA4, CTNNA2, CTNNB1, CTNND1, CTSC, CUL1, CUL3, CUL4A, CUL5, CUL7, CUL9, CXCR4, CYC1, CYP19A1, DAB2IP, DAND5, DAP3, DAPK1, DAXX, DBNL, DCN, DCTN2, DDB1, DEAF1, DEDD2, DEDD, DERL1, DGKA, DGKZ, DHCR24, DHFR, DLG2, DLG4, DLGAP3, DMP1, DMTF1, DNAJA3, DNAJB1, DNAJC5, DNMBP, DNMT1, DOCK4, DOK1, DPP4, DTL, DUSP14, DYNLL1, DYNLL2, DYRK1A, E4F1, ECSIT, EEF1A1, EEF2, EFEMP2, EGFR, EGR1, EHMT1, EHMT2, EIF2AK2, EIF2C2, EIF2C4, EIF3I, ELAVL1, ELK1, ELL, ELP2, ENPP1, ENSA, EP300, EP400, EPB42, EPHA3, EPOR, EPS15, EPS8L3, ERBB2IP, ERBB2, ERBB4, ERCC2, ERCC3, ERCC6, ERCC8, ERLIN2, ESR1, ETHE1, ETS1, ETS2, EWSR1, EZR, FADD, FAF1, FAF2, FAIM2, FAM82A2, FASLG, FASN, FAS, FBF1, FBXO11, FBXO42, FBXO45, FBXO4, FBXW11, FCGR3A, FCGR3B, FCN1, FCRL3, FGFR1, FGR, FKBP1A, FKBP3, FLII, FMOD, FN1, FNBP1, FNTA, FOXP3, FYB, FYN, G3BP1, G3BP2, G6PD, GAB2, GABARAPL1, GABBR1, GANAB, GAPDH, GBA, GEMIN4, GET4, GHR, GLG1, GLUL, GNAO1, GNB1, GNB2L1, GNB2, GNL3L, GNL3, GOLGA2, GP1BA, GPS1, GPS2, GPSM2, GRAP2, GRAP, GRB10, GRB2, GRK1, GRK5, GRK6, GSK3B, GTF2A1, GTF2H1, GTF2I, GZMA, GZMB, H2AFX, H3F3A, HABP4, HBS1L, HCK, HDAC1, HDAC2, HDAC4, HDAC5, HDAC6, HDAC7, HDAC9, HDC, HECA, HECTD3, HECW1, HECW2, HERPUD1, HEXIM1, HFE, HGF, HGS, HIF1A, HIPK1, HIPK2, HIPK3, HIST1H1A, HIST1H1B, HIST1H3A, HIST2H2AB, HIST2H2AC, HIST2H2BE, HIST2H3C, HIST3H3, HIST4H4, HK1, HLA-A, HLA-B, HLA-C, HLA-DRB1, HLA-E, HLA-G, HMGA1, HMGB1, HMGB2, HNF1A, HNF4A, HNRNPA2B1, HNRNPD, HNRNPH2, HNRNPK, HOXB1, HOXB3, HOXC6, HOXD10, HOXD11, HOXD3, HOXD8, HOXD9, HPR, HRAS, HSP90AA1, HSP90AB1, HSP90B3P, HSPA1A, HSPA1B, HSPA4, HSPA5, HSPA8, HSPA9, HSPB1, HSPB2, HSPB6, HSPB8, HTR2A, HTT, HUWE1, IARS, IBTK, ICAM1, ICAM5, IFI16, IFNAR2, IFN-γR1, IFN-γR2, IFN-γ, IGF1R, IKBKB, IKBKE, IKBKG, IKZF3, IKZF4, IL10RA, IL10, IL12RB2, IL13, IL1R1, IL1RAP, IL1RL1, IL21R, IL27RA, IL2RA, IL2RB, IL2RG, IL2, IL4R, IL4, IL5RA, IL5, IL6ST, IL9R, IMMT, INA, ING1, ING2, ING3, ING4, ING5, INSR, IRAK1, IRAK2, IRAK3, IRAK4, IRF1, IRF5, IRF7, IRF8, IRF9, IRS1, IRS2, ITCH, ITGAL, ITGAM, ITGB2, ITGB7, ITK, ITPK1, ITSN1, ITSN2, IVNS1ABP, JAK1, JAK2, JAK3, KALRN, KAT2A, KAT2B, KAT5, KCNA3, KCNAB1, KCNAB2, KCNH8, KDM1A, KDM4D, KEAP1, KHDRBS1, KIAA1191, KIR3DL2, KIT, KLRA1, KLRC1, KLRC2, KLRC3, KLRD1, KPNA1, KPNA2, KPNA4, KPNB1, KRAS, KRIT1, L3MBTL, LACTB, LAMP2, LATS2, LAT, LCK, LCP2, LGALS1, LGALS2, LGALS3BP, LGALS3, LILRB1, LILRB2, LLGL1, LMAN1, LMNA, LNX1, LRRC23, LRRFIP1, LRRFIP2, LRRK2, LSAMP, LTA, LTBP1, LTB, LY96, LYN, MACC1, MAD1L1, MADCAM1, MADD, MAFK, MAGEA1, MAGEA2, MAGEA4, MAGEA5, MAGED2, MAGEH1, MAP1A, MAP1B, MAP1LC3A, MAP2K1, MAP2, MAP3K14, MAP3K1, MAP3K5, MAP3K7, MAP3K8, MAP4, MAP6, MAP7D1, MAP9, MAPK10, MAPK14, MAPK1, MAPK3, MAPK8, MAPK9, MAPT, MARCH1, MARCH8, MARK1, MARK2, MARS, MAVS, MBD4, MDC1, MDH1, MDK, MDM2, MDM4, MECP2, MED12, MED14, MED16, MED17, MED1, MED21, MED24, MEF2D, MEN1, MEOX2, METAP1, METAP2, MGST3, MIA, MIF, MKRN1, MLL5, MLLT4, MLL, MMP2, MMP7, MMP9, MNAT1, MPDZ, MPP2, MPP4, MS4A1, MS4A2, MSH2, MSH6, MSL2, MSN, MTA1, MTA2, MTCH2, MTOR, MUC16, MUC1, MUL1, MYC, MYD88, MYH10, MYL12B, MYO15A, MYO5A, MYOC, MYST1, MYST2, MYST3, NANOS1, NANOS2, NCAM1, NCAN, NCF1, NCK1, NCK2, NCKIPSD, NCL, NCOA1, NCOA3, NCOA6, NCOR1, NDN, NDUFA10, NDUFA11, NDUFA12, NDUFA13, NDUFA2, NDUFA5, NDUFA6, NDUFA8, NDUFA9, NDUFB10, NDUFB11, NDUFB3, NDUFB4, NDUFB5, NDUFB6, NDUFB7, NDUFB8, NDUFB9, NDUFC2, NDUFS1, NDUFS2, NDUFS3, NDUFS4, NDUFS5, NDUFS7, NDUFS8, NDUFV1, NDUFV2, NEDD4, NEFL, NF1, NFATC1, NFATC2, NFKB1, NFKBIA, NFYA, NFYB, NIT2, NOC2L, NOD1, NOL3, NOS1AP, NOS1, NOS2, NOTCH1, NOTCH4, NPM1, NPPC, NQO1, NQO2, NR0B2, NR3C1, NR4A1, NRAS, NSMAF, NTM, NUB1, NUMB, NUP35, OBFC2B, OPA1, OPCML, OSTF1, OTUB1, PABPC1, PACSIN2, PADI4, PAEP, PAG1, PAK3, PARD6A, PARK2, PARK7, PARP1, PCGF3, PCNA, PD-L2, PD-1, PDCD5, PDCD6, PDE6D, PDIA2, PDIA3, PDLIM7, PDZD2, PELI1, PEX2, PGR, PHB2, PHB, PHF1, PHF20, PIAS1, PIAS2, PIK3CA, PIK3CD, PIK3CG, PIK3R1, PIK3R2, PIN1, PINK1, PIP4K2B, PLAGL1, PLAT, PLAUR, PLAU, PLCB2, PLCG1, PLD1, PLG, PLK1, PLK2, PLK3, PML, POLA1, POLDIP3, POLR1A, POLR2A, POMP, POR, POTEF, POU2F1, POU5F1, PPARA, PPARGC1A, PPIA, PPIL5, PPP1CA, PPP1CC, PPP1R13B, PPP1R13L, PPP2R1B, PPP2R4, PPP2R5A, PPP4C, PRAM1, PRDM2, PRF1, PRKCB, PRKCD, PRKCSH, PRKCZ, PRKD1, PRKDC, PRKRA, PRKRIR, PRLR, PRMT1, PRMT3, PRMT5, PSEN1, PSMA1, PSMA2, PSMA3, PSMA4, PSMA6, PSMA7, PSMB10, PSMB1, PSMB3, PSMB4, PSMB5, PSMB6, PSMB7, PSMB8, PSMB9, PSMC3, PSMC5, PSMD10, PSMD1, PSMD2, PSMD4, PSME1, PSME2, PSME3, PTAFR, PTEN, PTGS1, PTGS2, PTK2B, PTK2, PTPN11, PTPN12, PTPN13, PTPN1, PTPN22, PTPN6, PTPRCAP, PTPRC, PTPRZ1, PTTG1, PUM2, PXDN, PXN, PZP, RAB3A, RAB5A, RAB8A, RABAC1, RABGEF1, RAC1, RAC2, RAC3, RAD23A, RAD23B, RAD51, RAF1, RAG1, RAG2, RALA, RALBP1, RALGDS, RANBP2, RAP1GDS1, RARA, RARRES3, RASA1, RASD1, RASGRF1, RASGRF2, RASIP1, RASSF1, RASSF2, RASSF3, RASSF5, RB1CC1, RB1, RBBP5, RBCK1, RBX1, RCHY1, RELA, REL, REPS1, REXO2, RFC1, RFFL, RFWD2, RFWD3, RGL1, RGL2, RGL4, RHEB, RHOA, RIMBP3C, RIN1, RING1, RIPK1, RIPK2, RIPK3, RNASE1, RNF10, RNF115, RNF128, RNF135, RNF20, RNF216, RNF2, RNF31, RNF34, RNF43, RPA1, RPL11, RPL23, RPL26, RPL5, RPS10, RPS20, RPS3, RPS7, RRM2B, RRM2, RYBP, S100A4, S100A9, S100B, SAMM50, SAMSN1, SAP30, SARM1, SDC2, SEC14L2, SEC14L3, SEC61A1, SEL1L, SELE, SELL, SELPLG, SELP, SEMA4D, SENP3, SEPP1, SEPSECS, SEPT2, SEPT4, SEPT6, SERBP1, SERPINB9, SERPING1, SERPINH1, SETD2, SETD7, SETD8, SET, SFN, SFRS1, SH2B1, SH2B2, SH3BP2, SH3GL3, SH3KBP1, SH3PXD2A, SH3PXD2B, SH3RF2, SHARPIN, SHBG, SHB, SHC1, SHOC2, SIAH1, SIAH2, SIGIRR, SIN3A, SIN3B, SIRPA, SIRT1, SIT1, SIVA1, SKAP1, SKAP2, SKI, SKP2, SLA2, SLAMF1, SLA, SLC19A1, SLC1A2, SLC1A3, SLC25A11, SLC25A12, SLC25A13, SLC25A1, SLC25A22, SLC25A3, SLC38A4, SLC6A20, SLC6A2, SLC6A3, SLC6A4, SLC9A3R1, SMAD1, SMAD2, SMAD3, SMAD6, SMAD7, SMARCA4, SMARCB1, SMARCC1, SMARCD1, SMN1, SMURF1, SMYD2, SNAI1, SNAP91, SNCAIP, SNCA, SNCB, SNIP1, SNRPB, SNRPE, SNTA1, SNX17, SNX18, SNX33, SNX9, SOCS1, SOCS3, SOD2, SORBS3, SOS1, SOX4, SP1, SP2, SP3, SPI1, SPIN1, SPNS1, SPN, SPRY2, SPSB1, SPSB2, SPSB4, SPTA1, SPTAN1, SPTBN1, SPTB, SQSTM1, SRCAP, SRCIN1, SRC, SRGAP1, SRGAP2, SRGAP3, SRGN, SRI, STAM2, STAM, STAP2, STARD7, STAT1, STAT3, STAT5A, STAT5B, STAT6, STK11, STUB1, STX1A, STXBP1, SULT1A2, SUMO1, SUPT3H, SUPT7L, SUPV3L1, SYK, SYN1, SYNGAP1, SYP, SYT2, SYVN1, TAB1, TAB2, TAC4, TADA1, TADA2A, TADA3, TAF10, TAF1A, TAF1B, TAF1C, TAF1, TAF5L, TAF5, TAF6, TAF9, TANK, TAP1, TAP2, TAPBP, TARP, TAT, TBK1, TBP, TCEB1, TDG, TDP2, TEC, TEP1, TFAM, TFAP2A, TFAP2C, TFDP1, TGF-β1, TGF-β2, TGF-βR1, TGF-βR2, TGF-βR3, TGF-βRAP1, TGM1, THBS1, THRAP3, THRA, THRB, THY1, TIAM1, TICAM1, TICAM2, TINAGL1, TIRAP, TJP3, TLE1, TLN1, TLR1, TLR2, TLR3, TLR4, TLR5, TLR9, TMED9, TMEM85, TMOD2, TNFAIP3, TNFRSF10A, TNFRSF10B, TNFRSF10C, TNFRSF10D, TNFRSF11B, TNFRSF13B, TNFRSF18, TNFRSF1A, TNFRSF1B, TNFRSF4, DcR3, TNFRSF9, TNFSF10, TNFSF11, TNFSF13, TNFSF14, TNFSF15, TNF, TNIP1, TOLLIP, TOMM22, TOMM40, TOP1, TOP2A, TOP2B, TOPORS, TOR1A, TP53BP1, TP53BP2, TP53INP1, TP53RK, TP53, TP63, TP73, TPPP, TPT1, TRADD, TRAF1, TRAF2, TRAF3IP2, TRAF3, TRAF4, TRAF5, TRAF6, TRAM1, TRAP1, TRAT1, TRIAP1, TRIB3, TRIM21, TRIM24, TRIM25, TRIM27, TRIM28, TRIM39, TRIM3, TRIM54, TRIM69, TRPC1, TRPC4AP, TRPC4, TRPC5, TRRAP, TSC22D1, TSG101, TSHR, TTC1, TTK, TUBA1A, TUBA1B, TUBA4A, TUBB1, TUBB3, TUBB4, TUBB6, TUBB, TUB, TWIST1, TXNRD1, TXN, TYK2, TYROBP, UBC, UBD, UBE2A, UBE2B, UBE2D2, UBE2H, UBE2I, UBE2J1, UBE2M, UBE2N, UBE3A, UBE4A, UBE4B, UBL4A, UBR5, UCHL1, UCHL5, UHRF2, UIMC1, UQCR10, UQCRB, UQCRC1, UQCRC2, UQCRFS1, UQCRQ, USMG5, USO1, USP10, USP19, USP29, USP2, USP32, USP42, USP4, USP7, USP9X, UTP14A, VAPA, VAPB, VASN, VAV1, VCAM1, VCAN, VCP, VDAC1, VDAC2, VDAC3, VDR, VHL, VPRBP, VPS24, VPS28, VPS52, VRK1, VRK2, VTN, WAS, WDR5, WRN, WT1, WWOX, XPA, XPC, XRCC1, XRCC5, XRCC6, YBX1, YES1, YWHAE, YWHAG, YWHAQ, YWHAZ, YY1, ZAP70, ZBTB2, ZBTB7A, ZDHHC23, ZHX2, ZMIZ1, ZMIZ2, ZMYND11, ZNF148, ZNF367, ZNF395, ZNF420, ZNF668.

TCGA Breast Cancer Dataset:

RNA-Seq data of 1,065 breast cancer (BRCA) tumor samples were obtained from The Cancer Genome Atlas (TCGA) together, with 111 non-malignant adjacent normal tissue samples. Two data matrices were created: cancer matrix (1356*1065) and normal matrix (1356*111). The patients' clinical information was obtained as well from TCGA and matched with their genomic information.

Sequential Biclustering:

Biclustering, also known as block clustering, co-clustering, or two-way clustering, is the technique of simultaneously clustering the rows and columns of a matrix. In this example, biclustering shuffled rows (genes) and columns (patients) of the data matrix to generate clusters with a minimum variation of gene expression amongst a group of patients (intra-cluster variation) and maximum variation with other groups of patients (inter-cluster variation).

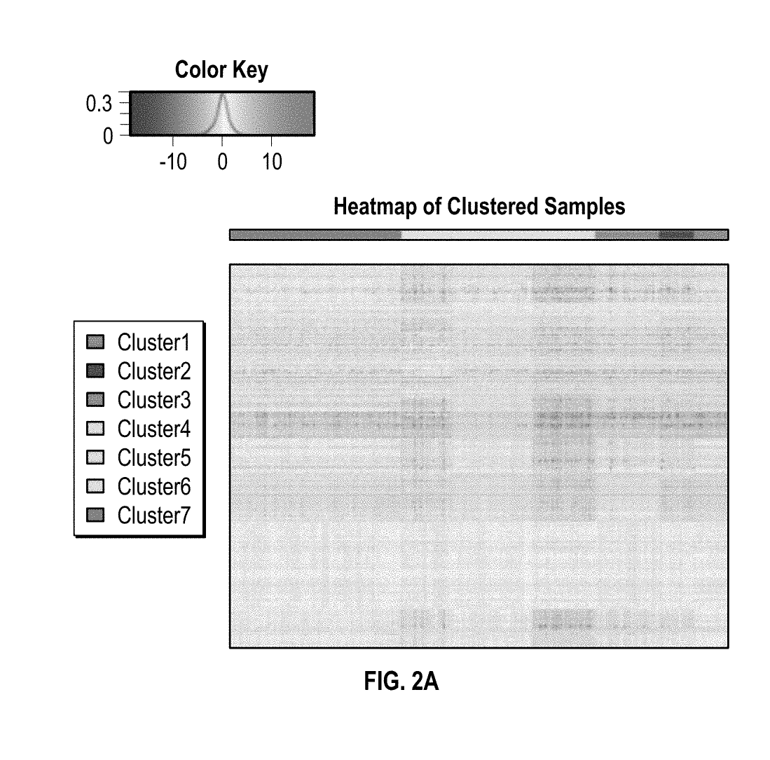

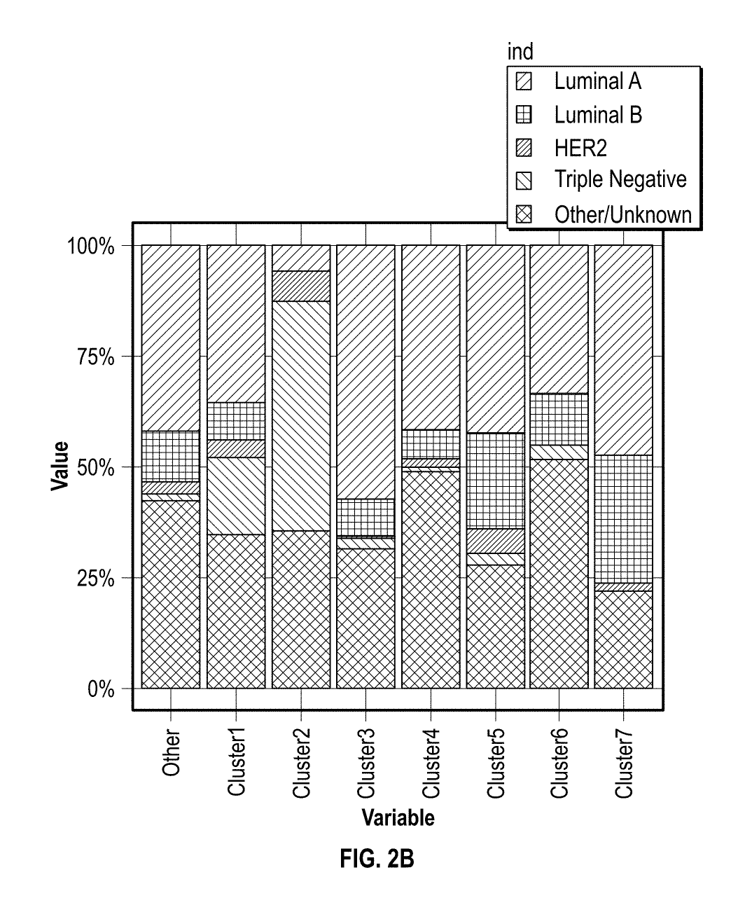

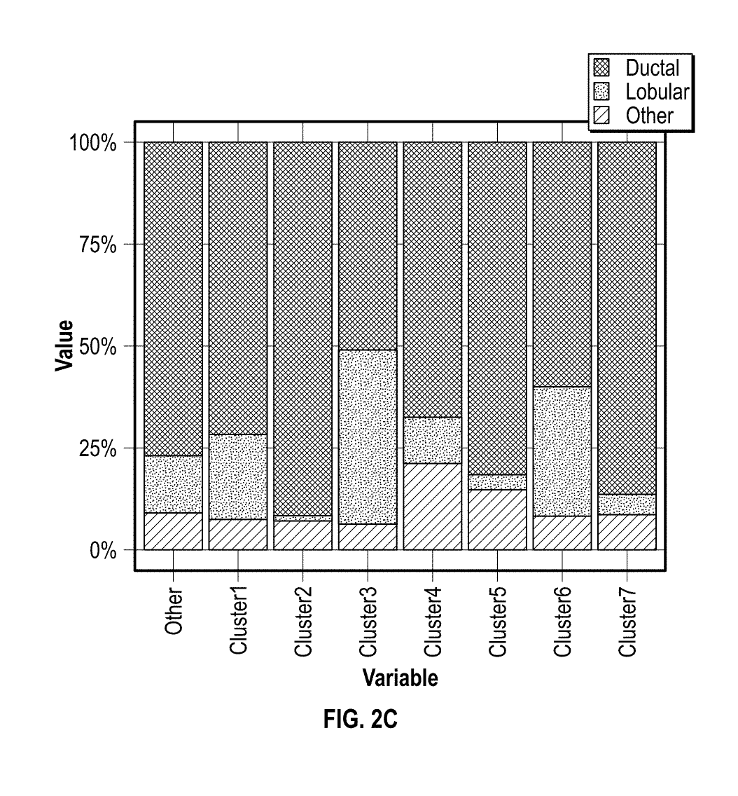

The biclust package available in R on the Log 2-transformed gene expression data of cancer patients was used to divide patients into subgroups based on their expression of immune-related genes. The BCPlaid algorithm was used as it clustered patients (columns) based on their similarity in gene expression (row-based) rather than by similar gene expression per patient (column-based)(FIG. 2A).

FIG. 2A, FIG. 2B, and FIG. 2C depicts the clustering results for the sequential biclustering of this example. FIG. 2A is a heatmap representing the level of expression of genes (rows) in different clusters of patients (columns). FIG. 2B depicts the percentage of different breast cancer receptor subtype, and FIG. 2C depicts lobular and ductal carcinomas, ILC and IDC, per cluster.

Running the BCPlaid algorithm generated overlapping clusters of patients and genes, where the clusters were sorted by layers output by the program. Each layer was searched based on residuals given all the previous layers, so by nature, earlier layers contained more information about the data and tended to be more coherent.

Since the goal of this example was to divide patients into non-overlapping groups, which meant that one patient should fall in only one group, a sequential approach was adopted. In this procedure, the BCPlaid algorithm was run multiple times sequentially. After each run, the earliest cluster was taken with at least 5% of the cancer patients and the patients in this cluster were removed from the whole dataset. The remaining dataset was then clustered in the next run.

The constraint of each candidate cluster having at least 5% of the cancer patients guaranteed that finally, each immune evasion cluster was a representative set of patients from the population.

Verification of Robustness of the Biclustering Procedure:

The robustness (reproducibility) of the proposed sequential biclustering procedure was assessed by running the whole procedure multiple times with five different random seeds. The procedure was re-run with each of these seeds and the agreement, measured by the random index, was checked between the clusters obtained by these runs and the original runs. Specifically, each seed resulted in a set of patient group labels C(i), i=1, 2 . . . , n, where n was the total number of patients. The original group labels were denoted as C₀(i), and the group labels from the other five runs were denoted as Cₖ(i), k=1, 2, 3, 4, 5. For each pair of patients 1≤i<j≤n, defined

$${δ}_{k}^{ij} = \{ \begin{matrix}1 , & \begin{matrix}if {C}_{0} ( i ) = {C}_{0} ( j ) , {C}_{k} ( i ) = {C}_{k} ( j ) {C}_{0} ( i ) \neq {C}_{0} ( j ) , \\ {C}_{k} ( i ) \neq {C}_{k} ( j )\end{matrix} \\ 0 , & otherwise\end{matrix} and \\ {γ}_{k} = \frac{{\sum }_{n \leq i \leq j \leq } {δ}_{k}^{ij}}{( \begin{matrix}n \\ 2\end{matrix} )} , k = 1 , 2 , 3 , 4 , 5.$$

This was an intuitive measure of consistency between two different sequences of biclustering runs, which was achieved by investigating if any two patients were clustered consistently. Values of agreement with the five random number seeds were all in the range of 90-93%, as depicted in the following table:

### Consistency Measures and Cross Validated Error Rates by a Classification Tree Model

| | | | | CV | | | Mean |
|---|---|---|---|---|---|---|---|
| | Consistency | CV error | CV error | error | CV error | CV error | error |
| | measure | rate 1 | rate 2 | rate 3 | rate 4 | rate 5 | rate |
| Seed 1234 | | 31.92% | 33.80% | 27.23% | 27.23% | 20.19% | 28.08% |
| Seed 655 | 92.17% | 34.74% | 35.21% | 27.69% | 31.92% | 30.51% | 32.01% |
| Seed 3016 | 90.08% | 30.51% | 31.45% | 30.51% | 30.98% | 37.56% | 32.20% |
| Seed 3877 | 91.60% | 31.92% | 30.04% | 32.86% | 29.10% | 36.15% | 32.01% |
| Seed 6918 | 93.18% | 29.10% | 27.70% | 32.64% | 30.04% | 30.99% | 30.09% |
| Seed 1574 | 92.09% | 32.39% | 33.33% | 33.80% | 31.92% | 33.33% | 32.95% |

Also compared were the results of each seed with the classification tree mentioned below, and error rates were relatively stable too, as indicated in the foregoing table.

Fisher Exact Test:

In this example, also determined was whether the clustering was dependent on breast cancer receptor status and invasive ductal and lobular subtypes. Accordingly, the receptor status information was obtained from TCGA and a Fisher exact test was used (see the following tables). The p-values were calculated by comparing the number of patients in a cluster belonging to a specific subtype to the total number of patients in the cluster. A p-value ≤0.05 indicated that distribution of a number of patients in that cluster was significantly different from the overall pattern.

The Association of Clusters with Breast Cancer Subtypes (Receptor Status)

| | | | | | | Fisher |
|---|---|---|---|---|---|---|
| | Number of | | | | | exact test |
| Cluster | patients | HER2 | Luminal A | Luminal B | TNBC | p-value |
| Cluster 1 | 193 | 12 (6.2%) | 105 (54.4%) | 25 (13.0%) | 51 (26.4%) | 8.46E−03 |
| Cluster 2 | 56 | 6 (10.7%) | 5 (8.9%) | 0 | 45 (80.4%) | 4.95E−26 |
| Cluster 3 | 98 | 1 (1%) | 82 (83.7%) | 12 (12.2%) | 3 (3%) | 3.10E−05 |
| Cluster 4 | 55 | 2 (3.6%) | 45 (81.8%) | 7 (12.7%) | 1 (1.8%) | 4.49E−03 |
| Cluster 5 | 80 | 6 (7.5%) | 47 (58.75%) | 24 (30%) | 3 (3.75%) | 9.30E−04 |
| Cluster 6 | 29 | 0 | 20 (68.9%) | 7 (24%) | 2 (6.9%) | 0.315 |
| Cluster 7 | 46 | 1 (2%) | 28 (60.9%) | 17 (37%) | 0 | 1.94E−04 |
| Other | 201 | 6 (3.0%) | 84 (41.8%) | 23 (11.4%) | 3 (1.5%) | 2.16E−04 |

HER2: Human epidermal growth factor receptor 2.

The results of the Fisher exact test showed a significant association between cluster 3, cluster 4, cluster 5, cluster 7 and Luminal A, and CL2 and triple negative breast cancer (TNBC) (P<0.05). Some patients had no information on their receptor status in TCGA.

The Association of Clusters with Invasive Lobular and Ductal Carcinoma Subgroups, ILC and IDC, Respectively.

| | Number of | | | | | Fisher exact |
|---|---|---|---|---|---|---|
| Cluster | patients | IDC | | ILC | | test p-value |
| Cluster 1 | 274 | 212 | (77.37%) | 62 | (22.63%) | 3.5030E−01 |
| Cluster 2 | 79 | 78 | (98.73%) | 1 | (1.27%) | 1.4315E−06 |
| Cluster 3 | 134 | 73 | (54.48%) | 61 | (45.52%) | 6.2561E−10 |
| Cluster 4 | 85 | 73 | (85.88%) | 12 | (14.12%) | 2.5155E−01 |
| Cluster 5 | 93 | 89 | (95.7%) | 4 | (4.30%) | 6.2294E−05 |
| Cluster 6 | 55 | 36 | (65.45%) | 19 | (34.55%) | 1.5219E−02 |
| Cluster 7 | 54 | 51 | (94.44%) | 3 | (5.56%) | 6.8520E−03 |
| Other | 182 | 154 | (84.62%) | 28 | (15.38%) | 1.8154E−01 |

Clusters 2, 5 and 7 were significantly associated with IDC and clusters 3 and 6 with ILC.

Differential Gene Expression Analysis:

Comparison of gene expression between any two groups was done by a combination of p-value from t-test and log 2 fold change cutoff. Both comparisons for tumor vs. tumor and tumor vs. normal were performed. Comparing tumor to tumor could be misleading as a gene may have been up-regulated, for example, in one tumor cluster compared to others but was yet less than normal.

Thus, it was important to use the normal as a reference. The number of genes per cluster may not always have been enough to help determine the mechanism of evasion. Knowing that each cluster of patients might have had different levels of gene expression, all the genes (rows) from the seven clusters were unified and checked for their mean expression per cluster of patients.

Pathway Analysis:

To find out whether an immune pathway was altered in each of the 7 clusters, the expression of genes in a cluster was compared with those of other clusters (tumor) and normal samples. The significantly differentially expressed genes and the corresponding KEGG pathways were plotted using the R/Bioconductor package, pathview for visualization (Luo, W. et al., Bioinformatics 2013; 29:1830-1. doi:10.1093/bioinformatics/btt285).

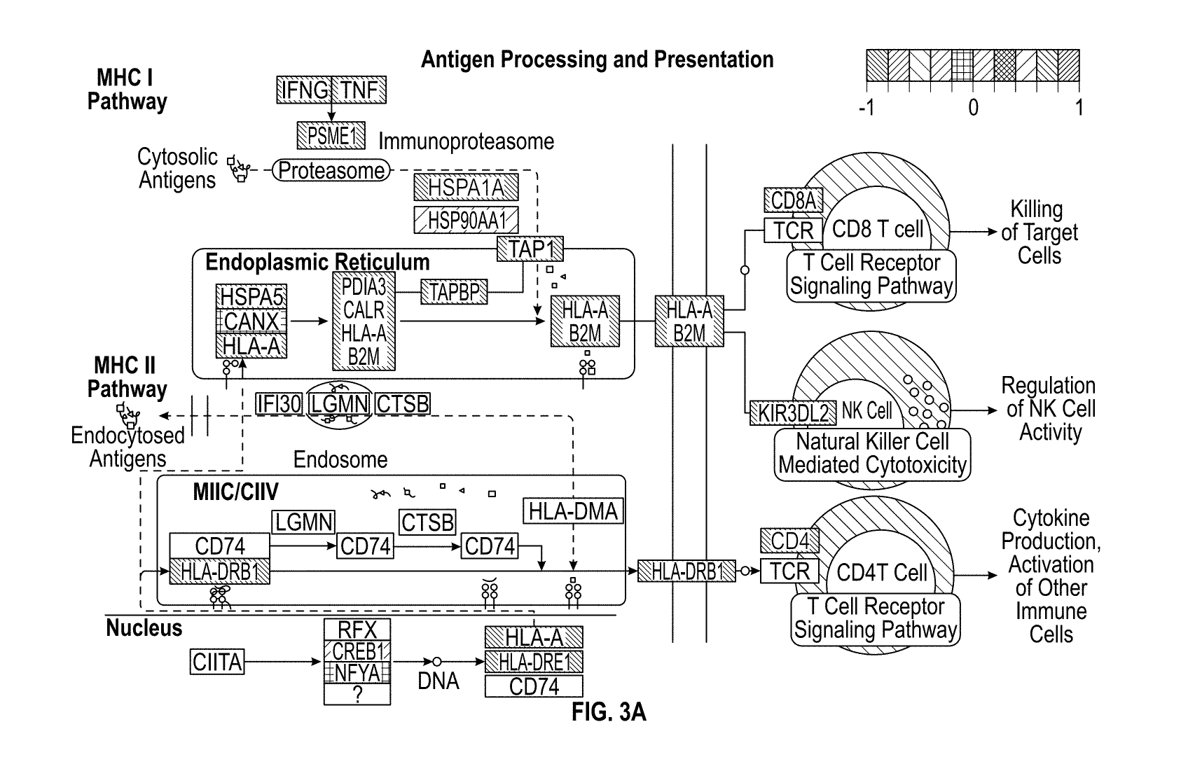

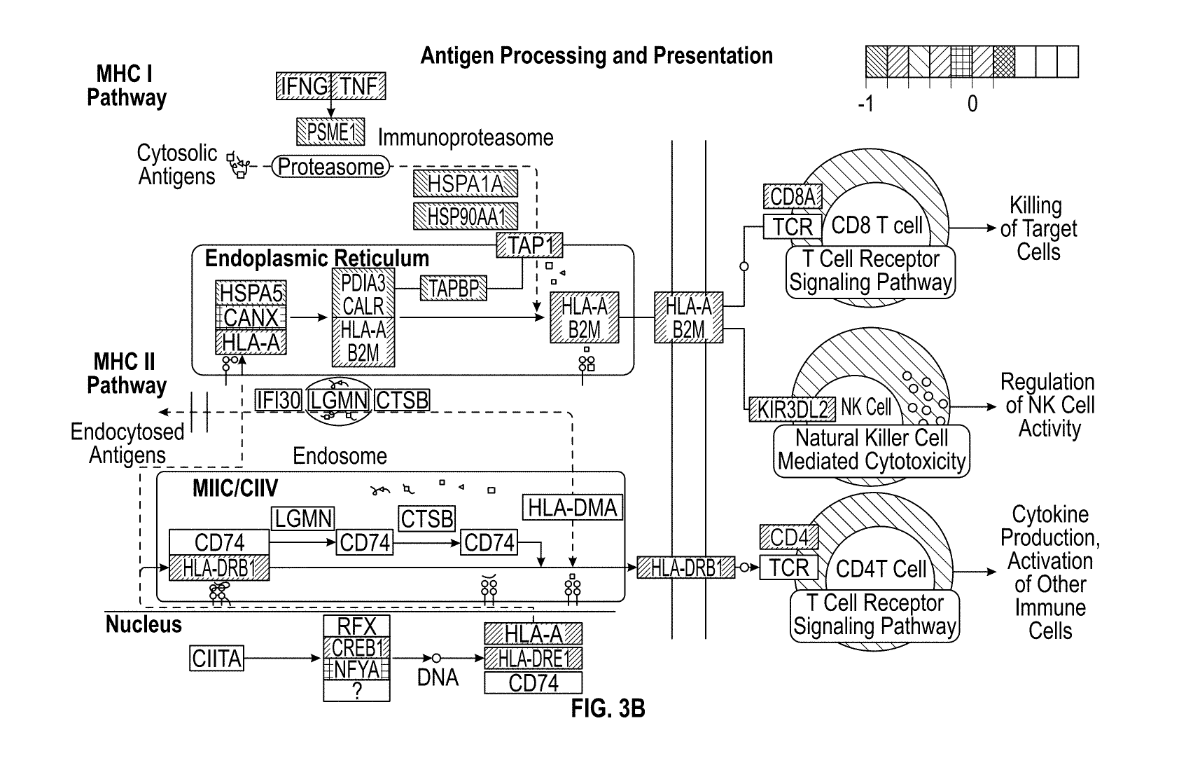

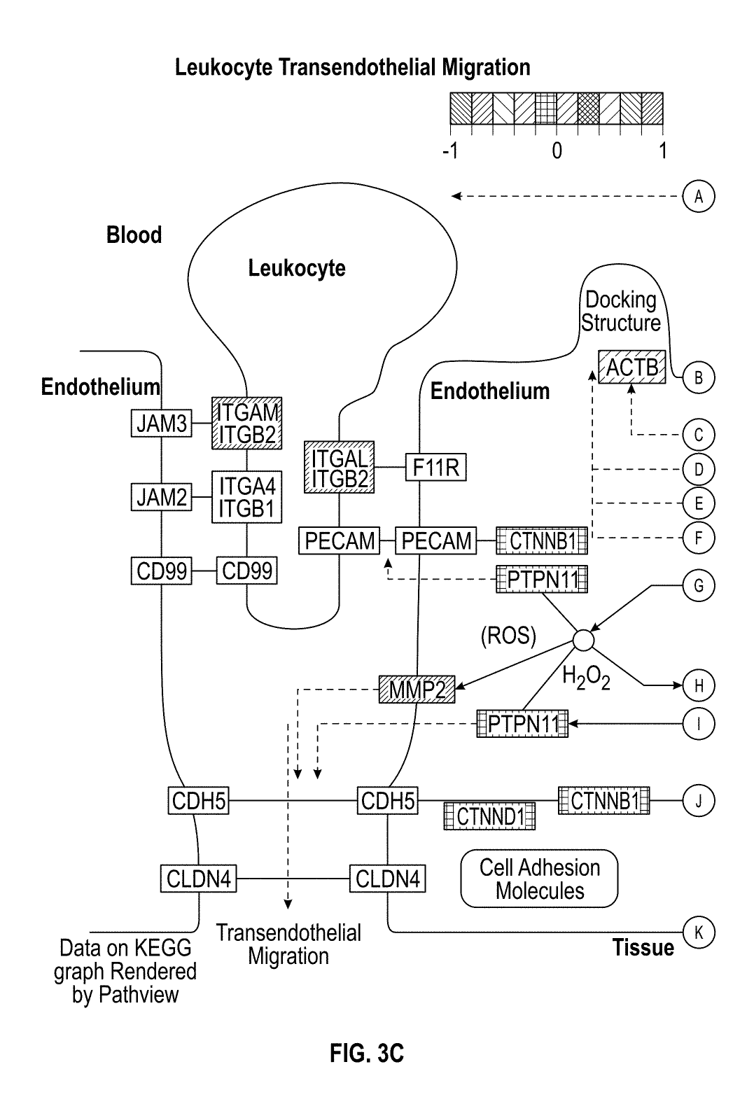

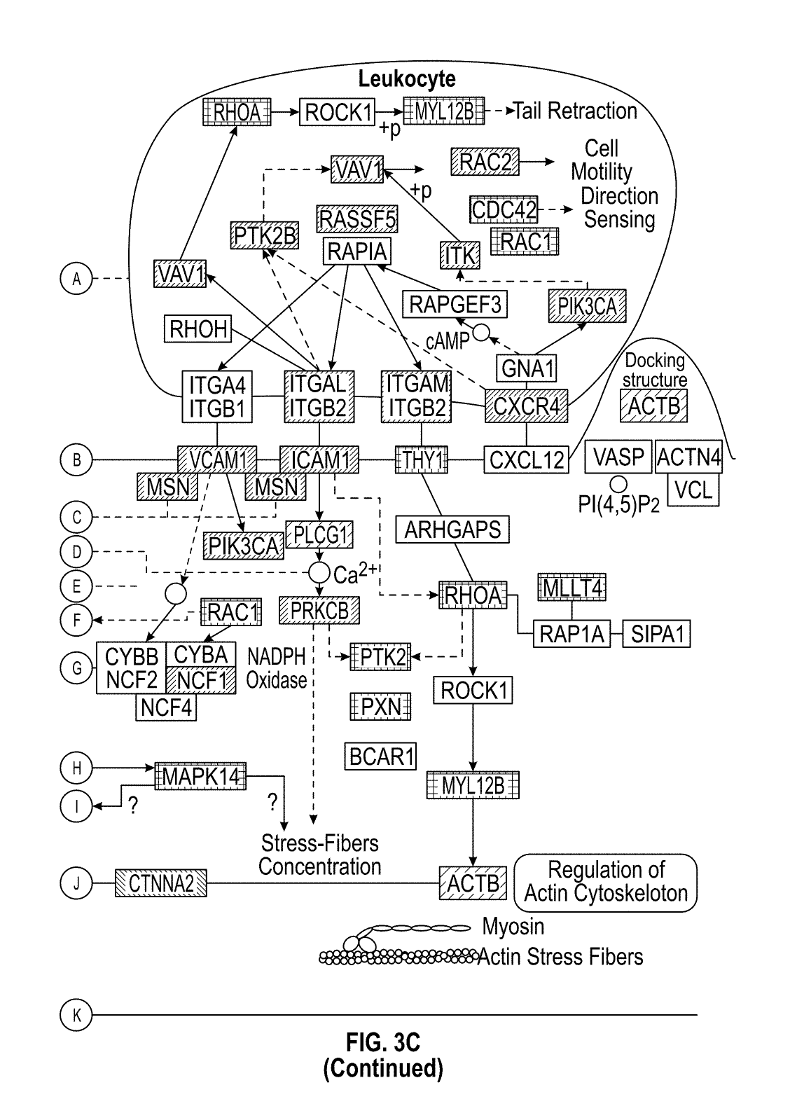

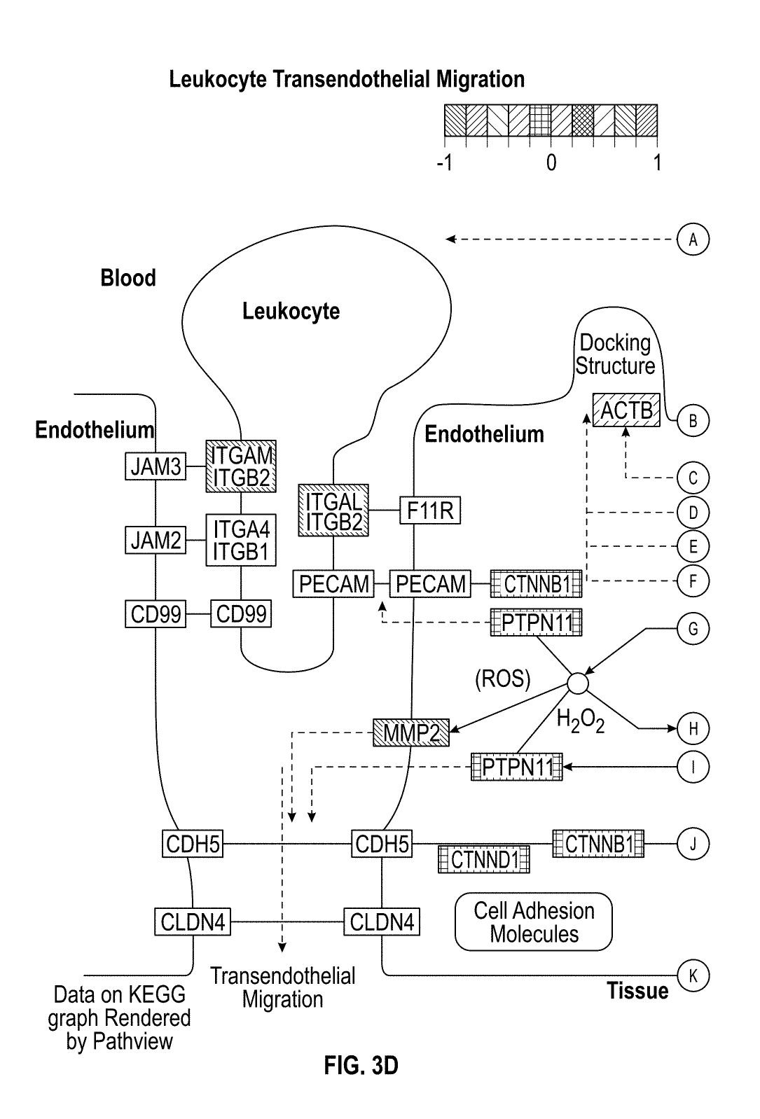

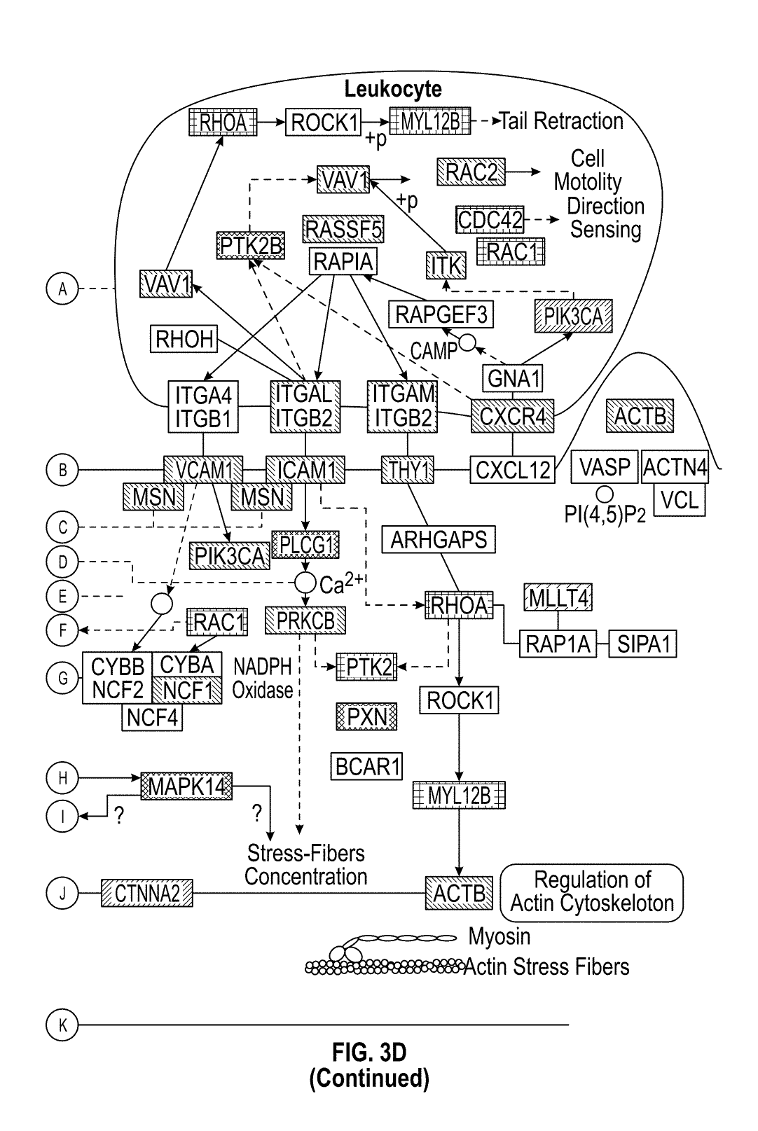

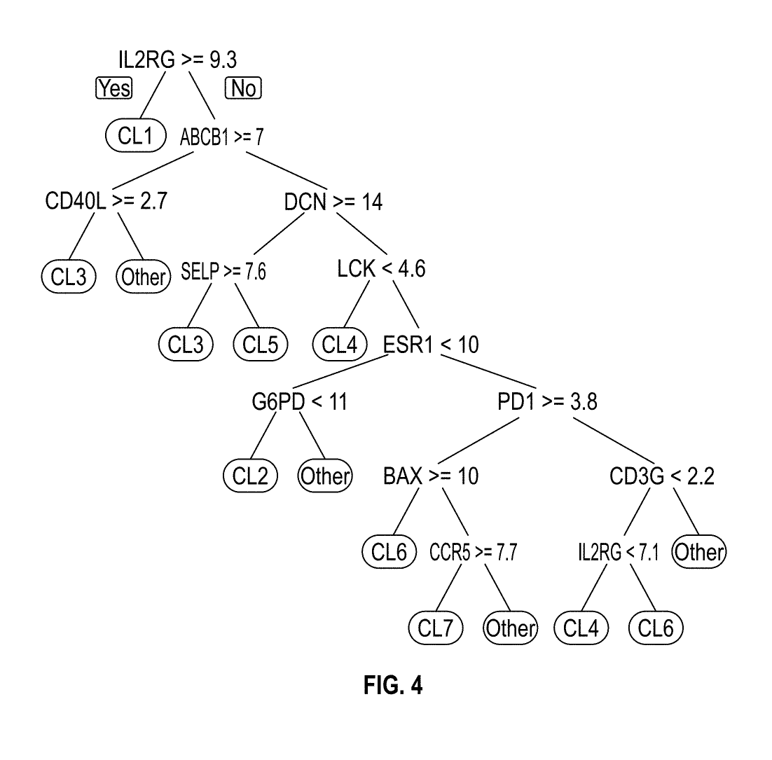

The immune-related pathways were chosen and showed only the pathways for antigen processing and presentation (hsa04612), leukocyte transendothelial migration (hsa04670), and cell adhesion molecules (hsa04514) that also showed the fold change of PD-1 (PDCD1), PD-L2 (PDCD1LG2), and CTLA4 (FIG. 3 and FIG. 4).

T-tests were implemented to compare the mean expression of a gene within a cluster to the mean expression of other cancer patients and to that of normal samples.

To analyze the t-test results, the genes were categorized into different groups based on their role in the cancer-immunity cycle, as depicted in the following table in which the groups are divided:

| Gene | Role |
|---|---|
| B2M | Beta-2 microglobulin |
| CALR | Calreticulin |
| HLA-A | Human leukocyte antigen A |
| HLA-B | Human leukocyte antigen B |
| HLA-DRB1 | Human leukocyte antigen DRB1 |
| HLA-E | Human leukocyte antigen E |
| PSMB8 | Proteasome subunit beta 8 |
| PSMB9 | Proteasome subunit beta 9 |
| TAP1 | Transporter 1 |
| TAP2 | Transporter 2 |
| TUBA4A | Tubulin alpha 4a |
| TUBB3 | Tubulin beta 3 class 3 |
| ICAM1 | Intercellular adhesion molecule 1 |
| ITGAL | Integrin Subunit Alpha L |
| ITGB2 | Integrin beta 2 |
| ITGB7 | Integrin beta 7 |
| SELL | Selectin L |
| SELP | Selectin P |
| SELPLG | Selectin P ligand |
| THY1 | Thymocyte antigen 1 |
| VCAM1 | Vascular cell adhesion molecule |
| CD80 | cluster of differentiation 80 |
| CD86 | cluster of differentiation 86 |
| CD4 | cluster of differentiation 4 |
| CD8A | cluster of differentiation 8A |
| GZMA | Granzyme A |
| GZMB | Granzyme B |
| IFN-γ | Interferon gamma |
| ITK | Interleukin-2-inducible T-cell kinase |
| KIR3DL2 | Killer Cell Immunoglobulin Like Receptor |
| KLRD1 | Killer cell lectin-like receptor D1 |
| PRF1 | Perforin 1 |
| ZAP70 | Zeta chain of T-Cell receptor associated protein kinase 70 |
| FAS | Apoptosis antigen 1 |
| FASLG | Apoptosis antigen 1 ligand |
| PD-1 | Programmed cell death protein 1 |
| PD-L1 | Programmed cell death protein 1 ligand 1 |
| PD-L2 | Programmed cell death protein 1 ligand 2 |
| BIRC3 | Baculoviral IAP repeat-containing protein 3 |
| TNFAIP3 | TNF alpha induced protein 3 |
| TRAF1 | TNF receptor associated factor 1 |
| TRAILR4 | TNF superfamily member 10 |
| BAK1 | BCL2 antagonist/Killer 1 |
| BAX | BCL2 associated X |
| BID | BH3 interacting domain death agonist |
| CASP10 | Caspase 10 |
| CCL22 | C-C motif chemokine 22 |
| CCR4 | C-C chemokine receptor type 4 |
| CTLA4 | Cytotoxic T lymphocyte-associated protein 4F |
| FOXP3 | Forkhead box P3 |
| IL10 | Interleukin 10 |
| IL10RA | Ilterleukin 10 receptor alpha |
| TGF-β1 | Tumor growth factor beta 1 |
| TNFRSF9 | Tumor necrosis factor receptor super family 9 |
| SERPINB9 | Serpin family B member 9 |
| TGF-β1 | Tumor growth factor beta 1 |
| TGF-β2 | Tumor growth factor beta 2 |
| TGF-βR1 | Tumor growth factor beta 1 receptor |
| TGF-βR2 | Timor growth factor beta 2 receptor |
| DcR3 | Decoy receptor 3 |

Using the generated p-values for the tumor-tumor and tumor-normal mean expressions, it was possible to understand at which level evasion was happening and using which molecules. FIGS. 3A-D depict the pathway analysis based on the log 2 fold change for clusters 1 and 4 compared to normal: fold change level of molecules involved in antigen processing and presentation molecules in cluster 1 (FIG. 3A) and cluster 4 (FIG. 3B). Fold change level of molecules involved in leukocyte recruitment in cluster 1 (FIG. 3C) and cluster 4 (FIG. 3D).

These results showed how cluster 1 genes for the first 2 steps of the cancer-immunity cycle were up-regulated while those of cluster 4 were mostly downregulated. The scale ranges from (−1 fold) downregulated expression, to the non-differential expression, to the (+1 fold) up-regulated expression.

Classification Tree:

After obtaining the 7 clusters, it was investigated whether it was possible to identify a small set of biomarker genes which could classify a subset of patients into their corresponding clusters. In addition, identifying potential biomarker genes would guide a deeper understanding of the different mechanisms of immune evasion subtypes, so that it would be possible to provide specific guidance on patient selection in combination therapy clinical trials and on personalized immunotherapy or combination of immunotherapies.

A classification tree was used to build a model to predict the cluster into which a patient sample belonged. To do this, the rpart package in R was used (Breiman, L. et al., Classification and Regression Trees. vol. 19. 1984; and Therneau, T. M. et al., “An introduction to recursive partitioning using the RPART routines,” Mayo Found 1997). The selected biomarker genes with their cutoff values are displayed at FIG. 4.

FIG. 4 depicts the classification tree of this example with 12 biomarkers and their log 2 gene expression cutoffs for the identified clusters (CL): Interleukin-2 receptor subunit gamma (IL2RG), ATP-binding cassette sub-family B member 1 (ABCB1), cluster of differentiation-40 ligand (CD40LG), decorin (DCN), lymphocyte-specific protein tyrosine kinase (LCK), selectin-P (SELP), estrogen receptor-1 (ESR1), glucose-6-phosphate dehydrogenase (G6PD), programmed cell death-1 (PDCD1), cluster of differentiation-3 subunit gamma.

Cohort and Patient Clustering:

To investigate the different evasion mechanisms in breast cancer, a list of 1,356 genes involved in immune activation and immune evasion was compiled as described herein. Then the RNA-seq expression data of these genes in breast cancer patients were obtained from TCGA database and used to categorize patients into different groups using a sequential biclustering algorithm based on BCPlaid (Lazzeroni, L. et al., Stat. Sin. 2002; 12:61-86). Eighty-one percent of TCGA breast cancer patients were clustered into 7 groups with non-overlapping patients, whereas the other nineteen percent fell into much smaller groups whose specific expression patterns were not characterized in this study. TCGA's 111 nonmalignant adjacent breast cancer samples were used as a normal reference for gene expression.

Identifying Evasion Mechanisms in the Seven Clusters:

To understand the mechanisms of evasion of patients falling into a specific group, the mean expression and fold change of genes involved in different steps of cancer-immunity cycle were compared to those of normal samples. The evasion mechanisms (M) were identified based on the rationale summarized in the following table, and discussed below.

### The Rationale for Deciding the Immune Evasion Mechanisms (M)

| | | Expression |
|---|---|---|
| | | compared to |
| Mechanism | Genes | normal |
| Immunosuppression | IL-10 or TGF-β1 or TGF-β2 | Up-regulated |
| (M1) | | |
| Tolerance (M2) | CTLA4 or (PD-1 and PD-L1/2) | Up-regulated |
| | or IFN-γ | |
| Antiapoptosis (M3) | At least 2 out of {BIRC3, | Up-regulated |
| | TNFAIP3, TRAF1, TRAILR4} | |
| Counterattack (M4) | DcR3 | Up-regulated |
| Impaired antigen | B2M or HLA-A or HLA-B | Not |
| presentation (M5) | | up-regulated |
| | and CD4 or CD8A | Not |
| | | up-regulated |
| | and at least 1 out of | Up-regulated |
| | {GZMA, GZMB, PRF1} | |
| | or TGF-β1 | Up-regulated |
| Ignorance (M6) | B2M and HLA-A and HLA-B | Not |
| | | up-regulated |
| | and CD4 and CD8A | Not |
| | | up-regulated |
| | and GZMA and GZMB and PRF1 | Not |
| | | up-regulated |
| | and TGF-β1 | Not |
| | | up-regulated |

### Abbreviations

Interleukin 10 (IL-10), tumor growth factor-beta (TGF-β1/2), cytotoxic T-lymphocyte associated protein 4 (CTLA4); programmed cell death-1 (PD-1) and ligand (PD-L1/2), interferon gamma (IFN-γ), Baculoviral IAP repeat-containing protein 3 (BIRC3), Tumor necrosis factor, alpha-induced protein 3 (TNFAIP3), TNF receptor-associated factor 1 (TRAF1), TNF-related apoptosis-inducing ligand receptor 4 (TRAILR4), decoy receptor 3 (DcR3); beta 2 microglobulin (B2M), human leukocyte antigen A and B (HLA-A/B), cluster of differentiation 4 (CD4), cluster of differentiation 8A (CD8A), granzyme A and B (GZMA/B), and perforin 1 (PRF1).

Patients in cluster 1 showed a high expression of molecules involved in antigen processing and presentation, leukocyte recruitment, and activation of immune cells compared to normal (FIG. 2A; FIG. 2B, Cluster 1). However, several immune-inhibitory molecules were up-regulated in this subtype. Molecules involved in immune tolerance (M2) such as CTLA4, PD-L1 and PD-1, and IFN-γ; and immune suppression (M1) such as IL-10, TGF-β1, and the Treg recruiting chemokine CCL22, were all significantly higher than normal. Tregs were recruited rather than induced in this subtype as the co-stimulatory tumor necrosis factor receptor TNFRSF9, which prevented the induction of naïve T-helper cells to Tregs (iTreg) by tumor microenvironment, was significantly up-regulated. Interferon gamma (IFN-γ) was among the highly-expressed genes in cluster 1 patients. Moreover, resistance to apoptosis (M3) manifested by the upregulated expression of anti-apoptotic molecules BIRC3, TRAF1, TNFAIP3, and the counterattack (M4) by DcR3, were unmissable.

The expression of antigen presenting molecules in cluster 2 was mostly up-regulated. HLA-A was up-regulated but both HLA-B and beta-2-microglobulin (B2M) were not differentially expressed from normal. To check if antigen presentation was impaired the downstream steps of the cancer-immunity cycle were examined. The expression of adhesion molecules was weak as almost all molecules were either not differentially expressed (ITGB2, ITGB7, THY1, and SELL) or significantly lower than the normal mean expression (ITGAL, VCAM, SELPLG, and SELP) (Cluster 2). The non-upregulated expression indicated that adhesion molecules were not stimulated, explaining the low recruitment of T-helper and CTL as their markers CD4 and CD8A were not up-regulated. Overall, this indicated that antigen presentation was impaired.

The expression of antigen presenting molecules in cluster 3 was mostly not higher than normal. Specifically, B2M, HLA-A, and HLA-B were not differentially expressed (Cluster 3). The low expression of MHC-I did not increase NK cell recruitment as the corresponding markers, KIR3DL2 and KLRD1, were not increased. The weak antigen presentation caused an inefficient recruitment of T-helper cells and CTLs (CD4 and CD8A) and deficient activation of CTL as a single cytotoxic molecule, GZMA, was up-regulated. TGF-β1 and DcR3 were up-regulated in this subtype. Thus, impaired antigen presentation (M5), immunosuppression (M1) and counterattack were the mechanisms of evasion in this subtype. The up-regulated expression of TGF-β1 (M1) decreased the expression of MHC-I and II molecules, inhibited the expression of B2M and HLA-DR, and resulted in decreased antigen presentation. TGF-β also inhibited CTL activation, proliferation, and differentiation; and the transcription of CTL's cytotoxic molecules, TCR components: ZAP70 and ITK. In addition, DcR3 (M4) increased invasion and migration of BRCA tumors, inhibited T cell activation and chemotaxis, and suppressed the activation and differentiation of dendritic cells (DCs) and macrophages, and altered the latter's phagocytic activity.

TNFRSF9, CCL22, and CCR4 were up-regulated but CTLA4 was not, thus, Tregs were not present. TNFRSF9 however, induced T cell apoptosis after activation (anergy) which was another mechanism to decrease CTL availability.

None of the cancer-immunity cycle steps were activated in Cluster 4. Thus, evasion in Cluster 4 was likely caused by the lack of a danger signal, aka ignorance.

The expression of molecules involved in the antigen presentation machinery was mostly not higher than normal in Cluster 5. Both T cell markers and CTL cytotoxic molecules were not up-regulated but TGF-β1 was (Cluster 5). Thus, evasion in Cluster 5 was potentially caused by TGF-β1-induced impairment of antigen presentation. The up-regulated expression of FOXP3 but not CTLA4 could be explained by increased expression of TGF-β1 and TNFRSF9. TGF-β inhibited CTL's activity by transforming naïve T helper cells to regulatory T cells (Tregs) by increasing naïve T helper expression of FOXP3. However, since TNFRSF9 prevented Treg induction by the tumor microenvironment, these T cells could not prohibit the CTLA4 marker. B2M in cluster 6 was not differentially expressed. Some leukocyte recruiting molecules were higher than normal (ITGAL, ITGB2 ITGB7, and THY1), whereas the rest were not upregulated, and all immune cell markers and cytotoxic molecules were not upregulated compared to normal (Cluster 6). This indicated that there was an impaired antigen presentation (M5) that resulted in impairment in leukocyte recruitment and subsequent activation.

CTLA4, TGF-β1, and DcR3 were higher than the normal mean expression, which indicated tolerance (M2), immunosuppression (M1), and counterattack (M4).

In Cluster 7 most of the genes involved in antigen presentation had a higher mean expression than normal. The expression of the majority of adhesion molecules was significantly higher than normal. CD4⁺ T-helper cells were not recruited to the tumor microenvironment whereas CD8⁺ CTLs were, as their marker (CD8A) and cytotoxic molecules were up-regulated. It seemed that Tregs were recruited to the tumor microenvironment as CTLA4, TNFRSF9, CCL22, and CCR4 were up-regulated (Cluster 7). IFN-γ had contradictory functions with either an anti-tumor or a pro-tumor effect. As an anti-tumor molecule, IFN-γ increased immune cell recruitment and caused a direct inhibition of tumor growth and recognition and elimination by the immune system. Other studies on IFN-γ pointed to a pro-tumor role, wherein it was shown to increase Treg development, decrease neutrophil infiltration, aide in tumor proliferation and resistance to apoptosis by CTL and NK cells, and increase PD-L1 expression. In both Clusters 1 and 7, Treg development was increased (CTLA4) and PD-L1 and anti-apoptotic molecules were up-regulated in cluster 1, showing a pro-tumor effect potentially caused by IFN-γ.

### LIST OF ABBREVIATIONS

B2M: Beta-2 microglobulin; BRCA: Breast Cancer; BIRC3: Baculoviral IAP repeat-containing protein 3; CCL22: C—C motif chemokine 22; CCR4: C—C chemokine receptor type 4; CD4: Cluster of differentiation 4; CD8: Cluster of differentiation 8; CTL: Cytotoxic T lymphocyte; CTLA4: Cytotoxic T lymphocyte-associated protein 4; DC: Dendritic cells; DcR3: Decoy receptor 3; FOXP3: Forkhead box P3; GZMA: Granzyme A; HLA-A: Major histocompatibility complex, class I, A; HLA-B: Major histocompatibility complex, class I, B; IFN-γ: Interferon gamma; IL-10: Interleukin 10; ITGB: Integrin beta; ITK: Interleukin-2-inducible T-cell kinase; KIR3DL2: Killer cell immunoglobulin-like receptor 3DL2; KLRD1: Killer cell lectin-like receptor subfamily D, member 1; MHC-I: Major histocompatibility complex, class I; NK: Natural killer; PD-1: Programmed cell death protein 1; PD-L1: Programmed cell death protein 1 ligand; SELP: P-selectin; SELPLG: Selectin P ligand; TCR: T-cell receptor; TGF-β: Transforming growth factor beta; TGF-β1: Transforming growth factor beta 1; TGF-β2: Transforming growth factor beta 2; THY1: Thymocyte antigen 1; TNFAIP3: Tumor necrosis factor, alpha-induced protein 3; TNFRSF9: Tumor necrosis factor receptor superfamily member 9; TRAF1: TNF receptor-associated factor 1; VCAM: Vascular cell adhesion molecule; ZAP70: Zeta-chain-associated protein kinase 70.

Evident Immunosuppression with TGF-β1 and TGF-β1+DcR3

Although several clusters had different combinations of evasion mechanisms, the majority of breast cancer patient groups shared and upregulated expression TGF-β1. Around 77.4% of the clustered TCGA breast cancer patients' evasion was through TGF-β1 immunosuppression, 57.7% with DcR3, 48.0% with CTLA4, and 34.3% with PD-1. All clusters with upregulated DcR3 (57.7%) had upregulated TGF-β1 expression as well, and all 34.3% with upregulated PD-1 had upregulated CTLA4 (see the following table). Thus, the most prevalent evasion mechanisms are through TGF-β1 and TGF-β1 and DcR3 combined.

DcR3 and TGF-β1 were shown to work in concert to induce epithelial to mesenchymal transitioning in colorectal cancer. Thus, it was possible that these molecules worked together in breast cancer as well to aid in tumor progression and immune evasion and serve as potential immunotherapy targets.

### The Mechanism of Evasion in Each Cluster and the Potential Immunotherapies for Future Clinical Trials

| | | Potential |
|---|---|---|
| Cluster | Mechanism of Evasion | Immunotherapies |
| Cluster 1 | M1: Immunosuppression: IL-10, | Anti-TGF-β1 |
| (TNBC) | TGF-β1 | Anti- PD-1 |
| | M2: Tolerance: CTLA4, PD-1/ | Anti-CTLA4 |
| | PD-L1, IFN-γ | Anti-DcR3 |
| | M3: Apoptosis resistance: Anti- | Anti-IFN-γ* |
| | apoptotic molecules^{¥} | |
| | M4: Counterattack: DcR3 | |
| Cluster 2 | M5: Impaired antigen | Ex-vivo modulated |
| (TNBC, IDC) | presentation | DCs + |
| | | Chemotherapy* |
| Cluster 3 | M1: Immunosuppression: TGF-β1 | Anti-TGF-β1 |
| (Luminal A, | M4: Counterattack: DcR3 | Anti-DcR3 |
| ILC) | M5: Impaired antigen | Ex-vivo modulated |
| | presentation | DCs |
| Cluster 4 | M6: Ignorance: No danger signals | Ex-vivo modulated |
| (Luminal A) | | DCs + |
| | | Chemotherapy* |
| Cluster 5 | M1: Immunosuppression: TGF-β1 | Anti-TGF-β1 |
| (Luminal B, | M5: Impaired antigen | Ex-vivo modulated |
| IDC) | presentation | DCs |
| Cluster 6 | M1: Immunosuppression: TGF-β1 | Anti-TGF-β1 |
| (ILC) | M2: Tolerance: CTLA4 | Anti-CTLA4 |
| | M4: Counterattack: DcR3 | Anti-DcR3 |
| | M5: Impaired antigen | Ex-vivo modulated |
| | presentation | DCs |
| Cluster 7 | M1: Immunosuppression: TGF-β1 | Anti-TGF-β1 |
| (Luminal B, | M2: Tolerance: CTLA4, IFN-γ | Anti-CTLA4 |
| IDC) | | Anti-IFN-γ* |

*require further investigation;
IDC: invasive ductal carcinoma;
ILC: invasive lobular carcinoma;
^{¥}These include BIRC3, TRAF1, TNFAIP3 and TRAILR4.

ILC and IDC Show Distinctive Evasion Combinations:

Forty-two percent of TCGA's ILC were significantly associated with clusters 3 and 6 while only 28% of IDC patients were significantly associated with clusters 2, 5 and 7. ILC was less common than IDC, however, studies have suggested that overall long-term outcomes of patients with ILC may be worse than those with stage-matched IDC and ILC patients can be less responsive to neoadjuvant chemotherapy.

An obvious distinction between the 2 subgroups was that IDC clusters had fewer combinations of mechanisms compared to ILC clusters and the counterattack via DcR3 (M4) was exclusive to ILC. Since certain chemotherapy treatments have immune-stimulating properties, they can help sensitize the immune response by promoting antigen presentation and tumor sensitization to T-cell mediated killing by Treg depletion. Thus, M1, M2 and M5 could be diminished by chemotherapy. This may be the reason why IDC derives greater benefit from chemotherapy compared to ILC which also evades via DcR3 and induces T cell apoptosis.

TNBC Fell into 2 Evasion Clusters:

TNBC was significantly associated with clusters 1 and 2, Luminal A with clusters 3 and 4, and Luminal B with clusters 5 and 7 (Table 1). TNBC patients were mostly distributed between cluster 1 which was identified with 4 different evasion mechanisms (47%) and cluster 2 where evasion was mainly driven by impaired antigen presentation (42%). Thus, there were 2 immune evasion subgroups for TNBC: one with only impaired antigen presentation and another with a combination of 4 evasion mechanisms. Although TNBC has been associated with poor prognosis, certain patients seemed to respond well to anthracycline-based chemotherapies.

Previous studies have shown that objective complete response was significantly associated with immune modules only in luminal breast cancer subgroups and that the tumor-infiltrating lymphocytes in TNBC had no significant interaction with paclitaxel plus non-pegylated liposomal doxorubicin-based therapies in neoadjuvant settings. In postneoadjuvant settings, tumor-infiltrating lymphocytes were associated with better prognosis in TNBC. Since chemotherapy may induce immunogenic cell death, it may be possible that TNBC patients falling into cluster 2 can overcome the impaired antigen presentation after chemotherapy-induced sensitization, resulting in the recruitment of TIL.

Furthermore, cluster 1 immune gene expression seemed to overlap with the immunomodulatory subtype of TNBC which was characterized by upregulated expression of genes involved in T cell function, interferon response, and antigen presentation.

Identification of Biomarkers for the Immune Evasion Clusters:

The discovery of seven immune evasion subtypes for breast cancer helped provide guidance for the choice of treatment. Using the molecules directly related to the immune evasion mechanisms, such as PD-L1, CTLA4, etc., may not be optimal especially since the results of this example showed that evasion likely occurred by a combination of several mechanisms and several molecules.

Instead, a decision tree was derived, 12 biomarkers were identified along with their gene expression thresholds (FIG. 4). Interestingly, the identified immune evasion genes were not all part of this set of putative biomarkers, which indicated that evasion genes may not be powerful in terms of identifying patients' evasion mechanisms.

The following table summarizes the classifier genes for each cluster and their corresponding functions.
Identified biomarkers using the classification tree algorithm for the seven breast cancer clusters (For each Cluster, the “Steps” indicate the order of the classifier gene looked at. The expression level is check and compared to other Clusters)

| | | | | | | Overall |
|---|---|---|---|---|---|---|
| | | | | | Implication of | Information |
| | | Classifier | Expression | | each classifier | Identified for |
| Cluster # | Order | gene | level | Compared to | gene | each Cluster |
| Cluster1 | 1 | IL2RG | Higher | Cluster2, | Higher growth | High growth |
| | | | | Cluster3, | and maturation | and maturation |
| | | | | Cluster4, | of lymphocytes | of lymphocytes |
| | | | | Cluster5, | | |
| | | | | Cluster6, | | |
| | | | | Cluster7, 0 | | |
| Cluster2 | 1 | IL2RG | Lower | Cluster1 | Lower growth | Low availability |
| | | | | | and maturation | of |
| | | | | | of lymphocytes | lymphocytes, T |
| | 2 | ABCB1 | Lower | Cluster3 (ABCB1 ≥ | Fewer T cell and | cell signaling, |
| | | | | 7) | APCs | and tumor |
| | 3 | DCN | Lower | Cluster3 (ABCB1 ≤ | Less tumor | growth |
| | | | | 7), Cluster5 | growth and | |
| | | | | | progression | |
| | 4 | LCK | Higher | Cluster4 | More TCR and | |
| | | | | | IL2 signaling | |
| | 5 | ESR1 | Lower | Cluster4, | Lower | |
| | | | | Cluster6, | availability of | |
| | | | | Cluster7 | immune cells | |
| | | | | | and weak | |
| | | | | | expression of | |
| | | | | | proinflammatory | |
| | | | | | cytokines and | |
| | | | | | type 1 | |
| | | | | | interferons | |
| | 6 | G6PD | Lower | 0 | Less sugar | |
| | | | | | metabolism, | |
| | | | | | Less growth | |
| Cluster3 | 1 | IL2RG | Lower | Cluster1 | Lower growth | Low T cell |
| | | | | | and maturation | recruitment |
| | | | | | of lymphocytes | and activation |
| | 2 | ABCB1 | Higher | Cluster2, | More T cells and | |
| | | | | Cluster4, | APCs | |
| | | | | Cluster5, | | |
| | | | | Cluster6, | | |
| | | | | Cluster7 | | |
| | 3 | CD40LG | Lower | 0 | Costimulates T- | |
| | | | | | cell proliferation | |
| | | | | | and cytokine | |
| | | | | | production | |
| | 2′ | ABCB1 | Lower | Cluster3 | Fewer T cells | |
| | | | | (others) | and APCs | |
| | 3′ | DCN | Higher | Cluster2, | More tumor | |
| | | | | Cluster4, | growth and | |
| | | | | Cluster6 | progression | |
| | 4′ | SELP | Higher | Cluster5 | More leuckocyte | |
| | | | | | recruitment | |
| Cluster4 | 1 | IL2RG | Lower | Cluster1 | Lower growth | Low T cell |
| | | | | | and maturation | availability, cell |
| | | | | | of lymphocytes | growth, |
| | 2 | ABCB1 | Lower | Cluster3 (ABCB1 ≥ | Fewer T cell and | maturation, |
| | | | | 7) | APCs | and activation |
| | 3 | DCN | Lower | Cluster3, | Less tumor | |
| | | | | Cluster5 | growth and | |
| | | | | | progression | |
| | 4 | LCK | Lower | Cluster2, | Less TCR and IL2 | |
| | | | | Cluster4, | signaling | |
| | | | | Cluster6 (PD-1 < | | |
| | | | | 3.8) | | |
| | 4′ | LCK | Higher | Cluster4 | More TCR and | |
| | | | | (others) | IL2 signaling | |
| | 5′ | ESR1 | Higher | Cluster2 | More availability | |
| | | | | | of immune cells | |
| | | | | | and enhanced | |
| | | | | | proinflammatory | |
| | | | | | medium and | |
| | | | | | type 1 interferon | |
| | | | | | expression | |
| | 6′ | PD-1 | Lower | Cluster6 (PD-1 > | Less T cell death | |
| | | | | 3.8), Cluster7 | | |
| | 7′ | CD3G | Lower | 0 | Low T cell | |
| | | | | | activation | |
| | 8′ | IL2RG | Lower | Cluster6 (rest) | Lower growth | |
| | | | | | and maturation | |
| | | | | | of lymphocytes | |
| Cluster5 | 1 | IL2RG | Lower | Cluster1 | Lower growth | High tumor |
| | | | | | and maturation | growth and |
| | | | | | of lymphocytes | progression. |
| | 2 | ABCB1 | Lower | Cluster3 (ABCB1 ≥ | Fewer T cells | Leuckocytes |
| | | | | 7) | and APCs | were recruited |
| | 3 | DCN | Higher | Cluster2, | More tumor | but had lower |
| | | | | Cluster4, | growth and | expression of |
| | | | | Cluster6 | progression | ABCB1 |
| | 4 | SELP | Lower | Cluster3 (ABCB1 ≤ | More leuckocyte | compared to |
| | | | | 7) | recruitment | Cluster3 |
| Cluster6 | 1 | IL2RG | Lower | Cluster1 | Lower growth | Low T cell |
| | | | | | and maturation | activation and |
| | | | | | of lymphocytes | more T cell |
| | 2 | ABCB1 | Lower | Cluster3 (ABCB1 ≥ | Fewer T cell and | death and |
| | | | | 7) | APCs | apoptosis |
| | 3 | DCN | Lower | Cluster3, | Less tumor | |
| | | | | Cluster5 | growth and | |
| | | | | | progression | |
| | 4 | LCK | Higher | Cluster4 (LCK < | More TCR and | |
| | | | | 4.6) | IL2 signaling | |
| | 5 | ESR1 | Higher | Cluster2 | More availability | |
| | | | | | of immune cells | |
| | | | | | and enhanced | |
| | | | | | proinflammatory | |
| | | | | | medium and | |
| | | | | | type 1 interferon | |
| | | | | | expression | |
| | 6 | PD-1 | Lower | Cluster6, | | |
| | | | | Cluster7 | | |
| | 7 | CD3G | Lower | 0 | Low T cell | |
| | | | | | activation | |
| | 8 | IL2RG | Lower | Cluster6 (PD-1 < | Lower growth | |
| | | | | 3.8) | and maturation | |
| | | | | | of lymphocytes | |
| | 6′ | PD-1 | Higher | Cluster4 (LCK ≥ | More T cell | |
| | | | | 4.6), Cluster6 | death | |
| | | | | PD-1 < 3.8) | | |
| | 7′ | BAX | Higher | Cluster7, 0 | More apoptosis | |
| Cluster7 | 1 | IL2RG | Lower | Cluster1 | Lower growth | Low T cell |
| | | | | | and maturation | availability, cell |
| | | | | | of lymphocytes | growth, |
| | 2 | ABCB1 | Lower | Cluster3 (ABCB1 ≥ | Fewer T cell and | maturation, |
| | | | | 7) | APCs | and activation; |
| | 3 | DCN | Lower | Cluster3, | Less tumor | and more T cell |
| | | | | Cluster5 | growth and | death (than |
| | | | | | progression | Cluster4 and |
| | 4 | LCK | Higher | Cluster4 (LCK < | More TCR and | Cluster6) and |
| | | | | 4.6) | IL2 signaling | apoptosis (than |
| | 5 | ESR1 | Higher | Cluster2 | More availability | Cluster6) |
| | | | | | of immune cells | |
| | | | | | and enhanced | |
| | | | | | proinflammatory | |
| | | | | | medium and | |
| | | | | | type 1 interferon | |
| | | | | | expression | |
| | 6 | PD-1 | Higher | Cluster4 (LCK ≥ | More T cell | |
| | | | | 4.6), Cluster6 | death | |
| | | | | (PD-1 < 3.8) | | |
| | 7 | BAX | Lower | Cluster6 (PD-1 > | More apoptosis | |
| | | | | 3.8) | | |
| | 8 | CCR5 | Higher | 0 | More leukocyte | |
| | | | | | trafficking | |

Tumors evade immune surveillance using 6 different mechanisms, which may occur simultaneously in the same tumor (FIG. 1). Thus, there were 63 possible combinations of mechanisms in BRCA. This heterogeneity even at the level of a single cancer hallmark was indicative of the challenge of identifying effective treatments.

Using the sequential biclustering method of this example on BRCA RNA-seq data from TCGA, seven clusters of BRCA patients were identified with different evasion mechanisms and combinations of mechanisms. The mechanisms of evasion were determined based on the expression of immune evasion genes and the genes involved in the cancer immunity cycle to aid in identifying potential immunotherapies for the corresponding subtypes.

To make it easier to identify patients' evasion group and thus, the choice of immunotherapy, the list of immune-related genes was narrowed to 12 biomarkers using the classification tree algorithm of this example. The classification tree was used due to its intuitive output with straightforward clinical interpretations. To further improve the classification accuracy, a random forest model was applied to the same data set.

The results indicated that there were different combinations of evasion mechanisms involved in breast cancer. It was found that 78.8% of the clustered patients evaded with TGF-β1-induced immunosuppression and 57.75% with a DcR3-induced counterattack. All clustered patients with upregulated DcR3 expression had TGF-β1 upregulated as well. Thus, targeting TGF-β1 alone or with DcR3 was believed to be promising for breast cancer.

The immunosuppressive nature of TGF-β has been well studied. It was shown that TGF-β disrupts antigen presentation and T cell activation, induces a Treg transformation from naïve T cells, and causes epithelial to mesenchymal transitioning. The blockade of TGF-β in colon cancer unleashed a potent and endured cytotoxic T-cell response against cancer cells, inhibited metastases, and rendered metastatic colon cancer more susceptible to anti-PD-1-PD-L1 therapy. DcR3 was shown to inhibit cytotoxicity against tumor cells and its expression was positively associated with cancer progression, angiogenesis, and metastasis. Furthermore, DcR3 was suggested as a prognostic factor for early tumor detection and a predictor of recurrence after resection in breast cancer, specifically.

Examining the invasive breast cancer subgroup, it was found that, compared to IDC, ILC-associated clusters had an exclusive upregulation of DcR3. This led to the hypothesis that the reason IDC benefited more from chemotherapy was that the latter helped diminish evasion by aiding in antigen presentation and killing Tregs. However, the upregulated expression of DcR3 in the ILC clusters resulted in further cytotoxic T cell death. The results contradicted a previous study on a Northeastern Chinese population that showed an increased expression of DcR3 in IDC. However, the population of this example was representative of a large racial and ethnic population.

Due to the higher immune system-tumor interactions involved, TNBC and HER2 were thought to be more immunogenic than Luminal A. However, the results of this example showed that TNBC patients split into two groups: the highly immunogenic cluster1 (TNBC: 51/105) and the less immunogenic (low leukocyte infiltration) cluster 2 (TNBC: 45/105). Based on the results of this example, it was hypothesized that cluster 1 TNBCs corresponded to the immunomodulatory TNBC subtype, and that cluster 2 TNBCs treatment with chemotherapy may trigger an immune response. Furthermore, the comparison between the different clusters showed that only cluster 1 was significantly associated with a higher proliferation gene expression signature. However, given the strong immune response in cluster 1, this may have been caused by the highly proliferating immune cells recruited to the tumor microenvironment rather than cancer cells' proliferation.

Currently, there are no clinical trials on DcR3 however, there are several ongoing for blockading CTLA4, TGF-β, PD1/PD-L1, CLTA4 and PD-1, and CAR-T. The list is summarized in the following table:

Cancer immunotherapies that may be used for breast cancer patients. Programmed cell death-1 (PD-1); cytotoxic T-lymphocyte associated protein 4 (CTLA4); decoy receptor 3 (DcR3); interferon gamma (IFN-γ); dendritic cell (DC); Chimeric antigen receptor T cells (CAR-Ts); tumor growth factor-beta 1 (TGF-β1)

| | Drug: | | Clinical |
|---|---|---|---|
| Treatment | Brand | | trial in |
| method | (generic) | FDA approved | breast cancer |
| anti-PD-1 | Opdivo | Melanoma, non-small | Yes |
| | (Nivolumab) | cell lung cancer, | |
| | | advanced lung cancer, | |
| | | metastatic renal cell | |
| | | carcinoma, classical | |
| | | Hodgkin's lymphoma, | |
| | | squamous cell | |
| | | carcinoma of head | |
| | | and neck, locally | |
| | | advanced or | |
| | | metastatic urothelial | |
| | | carcinoma | |
| | Keytruda | Metastatic melanoma, | Yes |
| | (Pembrolizumab, | metastatic non-small | |
| | MK-3475) | cell lung cancer, | |
| | | recurrent or | |
| | | metastatic head and | |
| | | neck cancer, | |
| | | refractory classical | |
| | | Hodgkin lymphoma, | |
| | | and metastatic | |
| | | urothelial carcinoma | |
| anti-CTLA4 | Yervoy ® | Late stage melanoma | Yes |
| | (Ipilimumab) | | |
| | Tremelimumab | No | Yes |
| Ex-vivo | Provenge | Metastatic castration- | No |
| modulated DCs | (Sipuleucel-T) | resistant PCA | |
| | Autologous | | Yes |
| | dendritic cell | | |
| | vaccination | | |
| Chimeric | Yescarta | Yes | Yes |
| antigen | (axicabtagene | | |
| receptor T | ciloleucel) | | |
| cells (CAR-Ts) | | | |
| anti-TGFB-1 | Fresolimumab | No | Yes |
| anti-DcR3 | To be developed | No | No |
| anti-IFN-γ | To be developed | No | No |

The response rate of blockading PD1/PD-L1 in breast cancer patients was tested in several hormone receptor subtypes. The overall response rate to PD-1 blockade in PD-L1 positive patients with advanced TNBC BRCA was 18.5% and 12% in ER+ and HER 2-advanced BRCA. The overall response rate to anti-PD-L1 was 24% in TNBC, and only 3% in metastatic BRCA −62.5% of whom were PD-L1+. In this latter study patients with TNBC experienced an overall response rate of 5.2%. No drugs targeting CTLA4 alone are currently in clinical trial. Combined anti-PD1 anti-CTLA-4 in eighteen patients with refractory metastatic BRCA resulted in an overall response rate of 17%: 0% for the 11 ER+ patients and 43% for the 7 TNBC patients. However, the small TNBC sample size made the results inconclusive. These results showed a poor overall response rate when the treatment criteria are based on breast cancer receptor subtypes and the expression of PD-1/PD-L1 and CTLA4, stressing the need for new criteria and more reliable biomarkers. The results of this example demonstrated that there was no one hormone receptor subtype that fell into one immune evasion cluster, rendering receptor subtyping a weak guide to the choice of immunotherapy. This example also showed that expression of PD-1 and CTLA4 was not a good biomarker for immunotherapy and hence the need for more reliable biomarkers.

Despite the large breast cancer sample size in TCGA, the absence of matched normal samples for about 90% of the breast cancer patients prevented the taking of individual genetic variation into account in this example. Moreover, the TCGA normal samples were not exactly normal but non-malignant adjacent samples.

Immune Related Gene List with Protein-Protein Interaction:

Based on the knowledge of the mechanism of tumor evasion from immune system destruction, a list of 87 genes was generated manually based on the available literature. The generated list included genes involved in the cancer-immunity cycle, and tolerance and immunosuppression-inducing genes. To make sure no important genes were missing, the list was expanded from 87 to 1,356 genes through adding all interacting proteins determined using bioGRID database (https://thebiogrid.org/). The data are publicly available from The Cancer Genomic Atlas (TCGA).

Sequential Biclustering:

Biclustering, also known as block clustering, co-clustering, or two-way clustering, is the technique of simultaneously clustering the rows and columns of a matrix. In some embodiments, biclustering shuffled rows (genes) and columns (patients) of the data matrix to generate clusters with minimum variation of gene expression amongst a group of patients (intra-cluster variation) and maximum variation with other groups of patients (inter-cluster variation). The biclust package available in R was used on the Log 2-transformed gene expression data of cancer patients to divide patients into subgroups based on their expression of immune genes.

The BCPlaid algorithm [SR1, SR2] was used as it clusters patients (columns) based on their similarity in gene expression (row-based) rather than by similar gene expression per patient (column-based). Running the BCPlaid generated overlapping clusters of patients and genes, where the clusters were ranked based on their degree of coherence of genes. Since the goal was to divide patients into groups, where one patient should fall in only one group, a sequential approach was adopted. In the examples herein, the BCPlaid algorithm was run multiple times sequentially. After each run, the top-ranked cluster with at least 5% of the cancer patients was taken, and the patients in this cluster were removed from the set of patients who were being clustered. The remaining patients were clustered in the next run. The constraint of each candidate cluster having at least 5% of the cancer patients guarantees that finally, each immune evasion subtype (IES) is a representative set of patients from the study population.

A General Differential Expression Function:

The convention that gene expressions are arranged in a matrix where each row represents a gene and each column represents a sample was followed. It was also assumed that input data was input after necessary normalization and on log 2 scale. For simplicity, it was recommend to pool two groups into a single matrix X. The input class label was a vector of length equal to the number of columns of X. It was either numeric, character, or factor, but it needed to have two unique values. The underlying procedure was a two-sided t-test. Test statistics and raw p-values were returned for further investigation. If the matrix X contained any missing value, the program continued implementing the tests and printed out notification which gene(s) have missing value(s).

Pathway Analysis:

To determine whether an immune pathway was altered in each of the 7 IES, expression of genes in an IES was compared with those of other IES (tumor) and normal samples. The significantly differentially expressed genes and the corresponding KEGG pathways were plotted using the R/Bioconductor package pathview for visualization. Immune related pathways were chosen. Only shown are the pathways for antigen processing and presentation (hsa04612), leukocyte transendothelial migration (hsa04670), and cell adhesion molecules (hsa04514) that also shows the fold change of PD-1 (PDCD1), PD-L2 (PDCD1LG2), and CTLA4 (FIGS. 2A-2D).

T-tests were implemented to compare the mean expression of a gene within an IES to the mean expression of other cancer patients and to that of normal samples. To analyze the t-test results, the genes were categorized into different groups based on their role in the cancer-immunity cycle. Using the generated p-values for the tumor-tumor and tumor-normal mean expressions, it was able to be understood at which level evasion was happening and using which molecules.

Heatmap Visualization of Cancer Data:

A heatmap was created for better visualization of the 7 clusters. The expressions used for this heatmap were standardized by the per-gene mean and standard deviation. Patients are ordered from Cluster 1 (left) to Cluster 7 (right), identified by the shaded bars above the heatmap.

Classification Tree with Cross-Validation:

After obtaining the 7 IES, it was investigated whether a small set of genes could be identified which could classify the patients into their corresponding IES. In addition, identifying potential biomarker genes could help with understanding more about the different mechanisms of immune evasion subtypes, so that specific guidance on patient selection in combination therapy clinical trials and on personalized immunotherapy or combination of immunotherapies could be provided.

A classification tree was implemented due to its intuitive output with straight forward clinical interpretations. This analysis was done by the rpart package in R. To validate the robustness of this procedure, a cross validation was also recommended to obtain cross-validated error rates. If the user decided not to implement any cross validation, a reminder message was printed.

User Prediction of Patient Subtype and Suggested Immunotherapy:

Based on the above classification strategy, a user-friendly function was incorporated that took any new (real or simulated) data as input and automatically outputted the patients' IES, related immune evasion mechanism(s), and suggested immunotherapy(ies). With the package, an R data frame immune table could be included that summarizes all the useful information for this part. The function also could allow users to develop their own immunotherapy table for different kinds of cancers.

Using the RNA-seq data of BRCA from The Cancer Genome Atlas (TCGA), seven IES were identified for 81% of the study population. These IES utilize 7 different combinations of the six evasion mechanisms (M1-M6): IES 1 (M1, M2, M3, M4); IES 2 (M5); IES 3 (M1, M4, M5); IES 4 (M6); IES 5 (M1, M5); IES 6 (M1, M2, M4, M5); and IES 7 (M1, M2), which represent 27:8%, 4:2%, 13:4%, 10:1%, 10:4%, 5:6% and 5:5% of the total patients, respectively. Approximately half of the triple negative breast cancer (TNBC) patients fell into subtype 1 and another half subtype 2. Biomarkers that helped classify a patient into one of the IES were also identified.

The tests of the examples provided herein unraveled a complex picture of immune evasion mechanisms of BRCA. The identified IES and their corresponding biomarkers provided guidance for rational design of combination immunotherapies in clinical trials by optimizing the choice of treatments and patient selection. In terms of statistical procedures, sequential biclustering, differential expression t-tests, classification and (cross validated) prediction were involved. The examples can be generalized to apply to different types of cancer and any gene list of interest.

Using the sequential biclustering method on BRCA RNA-seq data from TCGA, seven IES of BRCA patients were identified that utilize different evasion mechanisms or combination of mechanisms. The mechanisms of evasion were determined based on the expression of immune evasion genes and genes involved in the cancer immunity cycle, and shed light on immunotherapies for the corresponding subtypes.

Of the immunoinhibitory molecules in M1 was IFN-γ, which was up-regulated in IES 1 and IES 7. IFN-γ has contradictory functions with either an anti-tumor or a pro-tumor effect. As an anti-tumor molecule, IFN-γ increased immune cell recruitment and caused a direct inhibition of tumor growth and recognition and elimination by the immune system. Other studies on IFN-γ pointed to a pro-tumor role, wherein it was shown to increase Treg development, decrease neutrophil infiltration, aide in tumor proliferation and resistance to apoptosis by CTL and NK cells, and increase PD-L1 expression. In both IES 1 and IES 7, Treg development was increased (CTLA4) in both IES 1 and IES 7, and PD-L1 and anti-apoptotic molecules were up-regulated in IES 1, showing a pro-tumor effect that may be caused by IFN-γ.

Ignorance in IES 4 could have occurred due to cancer cells expression of a tumor associated antigen that was recognized by APCs as a “self” molecule. Patients in these subtypes can be treated by ex-vivo modulated DCs or CAR-Ts in combination with chemotherapy. Chemotherapy can help antigen recognition by increasing antigen release from dying cancer cells and eliciting an anti-tumor immune response.

A Fisher exact test [SR6, SR7, SR8] was also implemented between the patient IES and breast cancer subtype, and some interesting patterns were found. The identified immune evasion subtypes 1 and 6 were not significantly associated with any hormone receptor subtype (Luminal A, Luminal B, HER2+, TNBC). However, IES 3, 4, 5, and 7 were significantly associated with Luminal A and IES 2 with TNBC. Due to the higher immune system-tumor interactions involved, TNBC and HER2 were thought to be more immunogenic than Luminal A. However, the results showed that TNBC patients split into two groups: the highly immunogenic IES 1 (TNBC: 51=105=48:57%) and the less immunogenic (low leukocyte in ltration) IES 2 (TNBC: 45=105=42:86%), indicating that these TNBC patients should be treated differently. Both TNBC and Luminal A associated with different IES may indicate further heterogeneity and complexity of breast cancer.

Classification with Random Forest:

In this example, a classification tree was fit in order to find important biomarker genes to distinguish the 7 clusters, and assist the study of different immune evasion mechanisms. A classification tree was used due to its intuitive output with straightforward clinical interpretations. Due to the fact that many machine learning algorithms can be used for the purpose of clustering and classification, a classification tree may not be optimal, in some instances, in terms of prediction accuracy, statistical performance, or a combination thereof. Therefore, a random forest model also was used in this example.

A random forest, also known as a “random decision forest”, is a collection of multiple decision trees. It employs the idea of Bootstrap resampling (see, e.g., Efron, B. et al., An Introduction to the Bootstrap, CRC Press, 1994; and Efron, B. et al., Stat. Sci. 2003, 18: 135-40), with majority voting (for classification) and mean prediction (for regression). A random forest can, in some instances, overcome a problem of overfitting in a decision tree.

In this example, the implementation in the R package randomForest was used. Candidate biomarker genes were defined as the top features selected by a mean decrease in accuracy and Gini coefficient in the model.

The following table displays the complete confusion matrix.

Confusion Matrix of a random forest model fitting to the TCGA BRCA data. Rows in the table correspond to the true cluster labels and columns correspond to predicted labels.

| | Cluster1 | Cluster2 | Cluster3 | Cluster4 | Cluster5 | Cluster6 | Cluster7 | Other |
|---|---|---|---|---|---|---|---|---|
| Cluster1 | 288 | 3 | 5 | 0 | 0 | 0 | 0 | 0 |
| Cluster2 | 3 | 84 | 0 | 0 | 0 | 0 | 0 | 0 |
| Cluster3 | 8 | 0 | 121 | 0 | 2 | 2 | 0 | 10 |
| Cluster4 | 0 | 0 | 0 | 91 | 0 | 2 | 0 | 15 |
| Cluster5 | 6 | 3 | 2 | 3 | 88 | 0 | 0 | 9 |
| Cluster6 | 3 | 1 | 3 | 1 | 1 | 28 | 6 | 17 |
| Cluster7 | 12 | 2 | 3 | 0 | 1 | 1 | 24 | 16 |
| Other | 0 | 3 | 8 | 7 | 5 | 0 | 0 | 178 |

The following table indicates the per-cluster sensitivity and specificity.

### Per-Cluster Sensitivity and Specificity of the Random Forest Model.

| | Cluster1 | Cluster2 | Cluster3 | Cluster4 | Cluster5 | Cluster6 | Cluster7 | Other |
|---|---|---|---|---|---|---|---|---|
| Sensitivity | 0.9 | 0.875 | 0.852 | 0.892 | 0.907 | 0.848 | 0.8 | 0.726 |
| Specificity | 0.989 | 0.997 | 0.976 | 0.982 | 0.976 | 0.969 | 0.966 | 0.972 |

The overall accuracy of this model was 0.847 with a 95% confidence interval (0.824, 0.868).

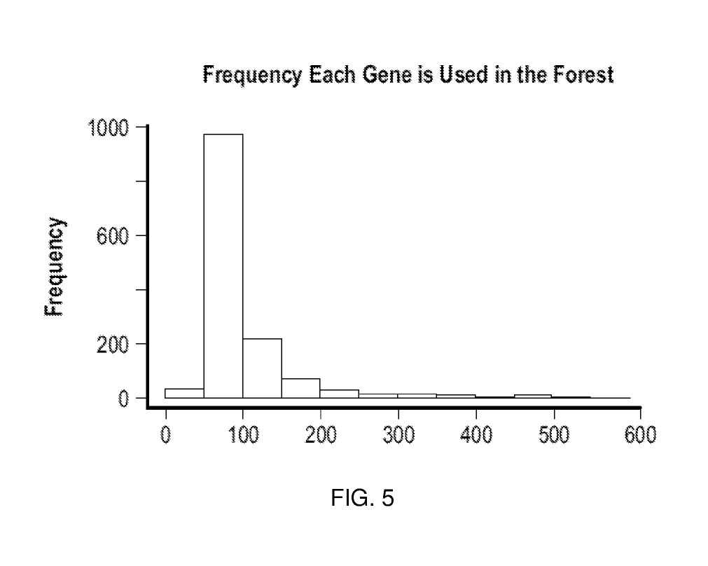

To visually investigate the quality of this model fitting, summary plots from the model were created. FIG. 5 is a histogram of the frequency of all genes being used in the model. With a forest with 1,000 trees, the right-skewed shape of the histogram indicated that a small proportion of genes were used much more than others, thereby indicating that those of the small proportion could serve as potential biomarkers.

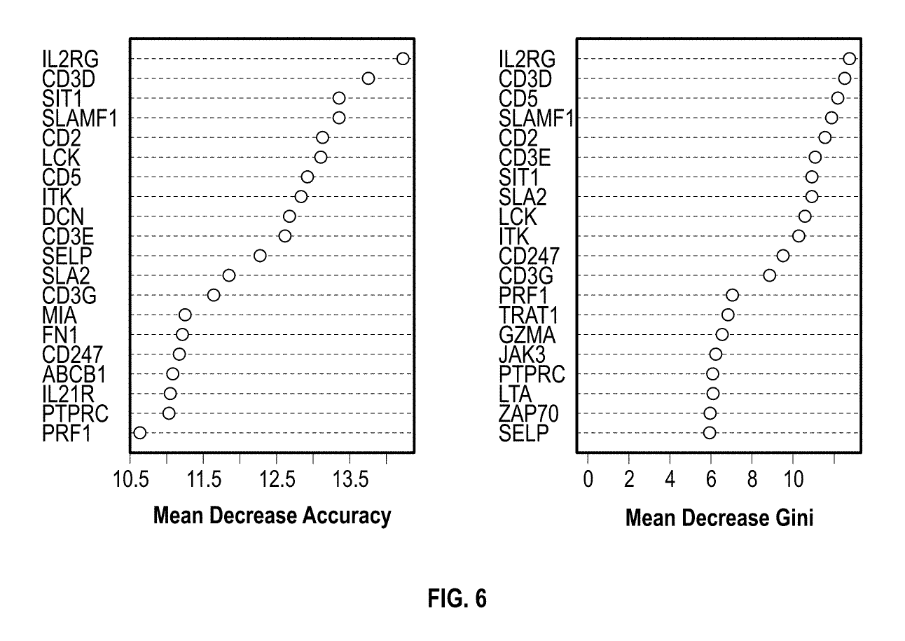

A more quantitative way to detect biomarker genes was performed, and the results are depicted at FIG. 6. FIG. 6 depicts plots of mean decrease in accuracy and mean decrease in Gini index for the 20 most important genes of this test. There was a substantial overlapping between the two lists.

There was also a high degree of agreeance with the biomarker selection performed by a single tree. As depicted at FIG. 4, IL2RG, ABCB1, DCN, LCK, and SELP were nodes on a higher level close to the root, and they all appeared to be important in the forest.

The success of the random forest test of this example indicated that it provides another option that may be used in the methods described herein.

We claim:

1. A method for treating a patient, the method comprising:
determining one or more expression levels for a set of biomarkers from a biological sample of the patient;
determining an immune evasion subtype for the biological sample based on the one or more expression levels;
selecting a treatment based on the immune evasion subtype; and
administering the treatment to the patient;
wherein the set of biomarkers comprises interleukin-2 receptor subunit gamma (IL2RG).

2. The method of claim 1, wherein the set of biomarkers further comprises at least one of ATP-binding cassette sub-family B member 1 (ABCB1) or decorin (DCN).

3. The method of claim 2, wherein the set of biomarkers further comprises at least one of lymphocyte-specific protein tyrosine kinase (LCK) or estrogen receptor-1 (ESR1).

4. The method of claim 3, wherein the set of biomarkers further comprises at least one of selectin-P (SELP), glucose-6-phosphate dehydrogenase (G6PD), programmed cell death-1 (PD-1), cluster of differentiation-3 subunit gamma (CD3G), B-Cell CLL/Lymphoma 2 (BCL2)-associated X Protein (BAX), C—C chemokine receptor type 5 (CCR5), or cluster of differentiation-40 ligand (CD40LG).

5. The method of claim 1, wherein the treatment comprises an anti-PDCD1 agent, an anti-cytotoxic T-lymphocyte-associated protein 4 (anti-CTLA4) agent, ex vivo modulated dendritic cells, chimeric antigen receptor T cells (CAR-Ts), an anti-transforming growth factor beta 1 (TGFβ-1) agent, an anti-decoy receptor 3 (DcR3) agent, an anti-interferon gamma (IFN-γ) agent, or a combination thereof.

6. The method of claim 1, wherein the treatment comprises an immunotherapy agent selected from anti-TGF-β1, anti-PD-1, anti-CTLA4, anti-DcR3, anti-IFN-γ*, ex-vivo modulated dendritic cells, or a combination thereof.

7. The method of claim 1, wherein the administering of the treatment comprises injecting the treatment intravenously.

8. The method of claim 1, wherein the biological sample comprises breast cancer tissue.

9. A method for treating a patient, the method comprising:
determining one or more expression levels for a set of biomarkers from a biological sample of the patient;
determining an immune evasion subtype for the biological sample based on the one or more expression levels;
selecting a treatment based on the immune evasion subtype; and
administering the treatment to the patient;
wherein the set of biomarkers comprises estrogen receptor-1 (ESR1).

10. The method of claim 9, wherein the set of biomarkers further comprises interleukin-2 receptor subunit gamma (IL2RG).

11. The method of claim 10, wherein the set of biomarkers further comprises at least one of selectin-P (SELP), signaling lymphocytic activation molecule family member 1 (SLAMF1), lymphocyte-specific protein tyrosine kinase (LCK), cluster of differentiation 2 (CD2), or casein kappa protein coding gene (CSN3).

12. The method of claim 11, wherein the set of biomarkers further comprises at least one of ATP-binding cassette sub-family B member 1 (ABCB1), decorin (DCN), cluster of differentiation-3 subunit gamma (CD3G), cluster of differentiation 5 (CD5), granzyme A (GZMA), or granzyme B (GZMB).

13. The method of claim 12, wherein the set of biomarkers further comprises at least one of cluster of differentiation-40 ligand (CD40LG), glucose-6-phosphate dehydrogenase (G6PD), programmed cell death-1 (PD-1), T-cell receptor T3 delta chain (CD3D), B-Cell CLL/Lymphoma 2 (BCL2)-associated X Protein (BAX), or C—C chemokine receptor type 5 (CCR5).

14. The method of claim 13, wherein the set of biomarkers further comprises at least one of tumor protein p63 (TP63), bone marrow tyrosine kinase gene in chromosome X protein (BMX), polo like kinase 1 (PLK1), transforming growth factor beta 2 (TGFB2), NADH ubiquinone oxidoreductase subunit B9 (NDUFB9), transforming growth factor beta receptor associated protein 1 (TGFBRAP1), 5-hydroxytryptamine receptor 2A (HTR2A), PLAG1 like zinc finger 1 (PLAGL1), sprouty RTK signaling antagonist 2 (SPRY2), protein tyrosine phosphatase receptor type C (PTPRC), Fc receptor like 3 (FCRL3), protein kinase C beta (PRKCB), heat shock protein beta-6 (HSPB6), contactin 1 (CNTN1), crystallin alpha B (CRYAB), heat shock protein 90 alpha family class A member 1 (HSP90AA1), MAGE family member A1 (MAGEA1), or interferon regulatory factor 8 (IRF8).

15. The method of claim 1, wherein the biological sample comprises breast cancer tissue.

16. A method for treating a patient, the method comprising:
determining one or more expression levels for a set of biomarkers from a biological sample of the patient;
determining an immune evasion subtype for the biological sample based on the one or more expression levels;
selecting a treatment based on the immune evasion subtype; and
administering the treatment to the patient;
wherein the set of biomarkers comprises interleukin-2 receptor subunit gamma (IL2RG).

17. The method of claim 16, wherein the set of biomarkers further comprises at least one of ATP-binding cassette sub-family B member 1 (ABCB1), signaling lymphocytic activation molecule family member 1 (SLAMF1), cluster of differentiation 2 (CD2), cluster of differentiation 5 (CD5), decorin (DCN), cluster of differentiation-3 subunit gamma (CD3G), selectin-P (SELP), or lymphocyte-specific protein tyrosine kinase (LCK).

18. The method of claim 17, wherein the set of biomarkers further comprises at least one of T-cell receptor T3 delta chain (CD3D), signaling threshold-regulating transmembrane adapter 1 (SIT1), IL2 inducible T cell kinase (ITK), CD3e molecule (CD3E), Src like adaptor 2 (SLA2), CD247 molecule (CD247), or granzyme A (GZMA).

19. The method of claim 16, wherein the treatment comprises an immunotherapy agent selected from anti-TGF-β1, anti-PD-1, anti-CTLA4, anti-DcR3, anti-IFN-γ*, ex-vivo modulated dendritic cells, or a combination thereof.

20. The method of claim 16, wherein the biological sample comprises breast cancer tissue.
